(19) United States
(12) Patent Application Publication
(10) Pub. No.: US 2019/0311787 A1
(43) Pub. Date: Oct. 10, 2019

(54) USER INTERFACE WITH DYNAMIC DISPLAY OF MATCHING CLINICAL TRIALS AS A PATIENT ANSWERS MORE QUESTIONS

(71) Applicant: ANTIDOTE TECHNOLOGIES LTD., London (GB)

(72) Inventors: Pablo GRAIVER, London (GB); Zeshan GHORY, London (GB); Anthony FINCH, London (GB); Jason MCFALL, London (GB); Duncan ROBERTSON, London (GB); Ruan KENDALL, London (GB); Dean SELLIS, London (GB)

(21) Appl. No.: 16/298,203

(22) Filed: Mar. 11, 2019

Related U.S. Application Data
(63) Continuation of application No. 15/790,818, filed on Oct. 23, 2017, now abandoned.
Application No. 15/790,818 is a continuation of application No. PCT/GB2016/051140, filed on Apr. 22, 2016.
(60) Provisional application No. 62/150,958, filed on Apr. 22, 2015.

(30) Foreign Application Priority Data
Apr. 22, 2015 (GB) 1506824.0

(51) Int. Cl. G16H 10/20; G06F 17/28; G16H 10/60; G06F 17/27

(52) U.S. Cl. CPC G16H 10/20; G06F 17/2705; G16H 10/60; G06F 17/2818

(57) ABSTRACT

The invention relates to systems for structuring clinical trials protocols into machine interpretable form. A hybrid human and natural language processing system is used to generate a structured computer parseable representation of a clinical trial protocol and its eligibility criteria. Furthermore, a web-based search engine to allow patients to find relevant clinical trials is developed. It works by asking a series of questions, which are generated dynamically such that previous answers will decide which question is generated next. Using a probabilistic model of trial suitability, questions are prioritized so as to minimize the total question burden. Furthermore, data collected across multiple trials is used to optimize the model and to optimize the design of future clinical trials.

### CROSS-REFERENCE TO RELATED APPLICATIONS

This is a continuation of U.S. application Ser. No. 15/790,818, filed on Oct. 23, 2017, which is a continuation of International Application No. PCT/GB2016/051140, filed Apr. 22, 2016, which claims priority to GB Application No. GB1506824.0, filed Apr. 22, 2015, and U.S. Provisional Application No. 62/150,958, filed Apr. 22, 2015, the entire contents of each of which being fully incorporated herein by reference.

### BACKGROUND OF THE INVENTION

### 1. Field of the Invention

The invention relates to a computer implemented method for determining clinical trial suitability or relevance. Implementations include methods and systems for structuring clinical trial protocols into machine interpretable form, methods and systems for interactively matching patient with suitable clinical trial, and methods and systems for aggregating data across multiple clinical trials.

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### 2. Technical Background

Clinical trial protocols that are available in the public domain are often very hard to understand for patients without a medical background as they have been designed for healthcare professionals. In particular, clinical trial eligibility criteria expressed using plain text are technically difficult to understand and further include complicated grammar and punctuations. From the plain text describing clinical trial protocols, it can be very difficult to extract information such as eligibility criteria or medical conditions for which a trial is suited.

Often, due to circumstances beyond the patient's control, patients fail to qualify for a clinical trial at the last part of the process, the site-based screening process. A common reason for this screening failure is poor quality (false positive) patients being sent to the sites through broad advertisements or superficial pre-screening.

A problem facing clinical trials is the recruitment of suitable candidates in order to meet a sample size requirement, such that the sample size of suitable candidates also represents adequately the targeted population. While patient interest and willingness is growing, the research ecosystem does not engage patients well, from the patient point-of-view and does not enable a streamlined process to consent and joining a clinical trial.

### 3. Discussion of Related Art

Typically, patients are recruited for clinical trials one trial at a time, for example by a Contract Research Organization working on behalf of a specific trial sponsor. This is often a manual process as there are currently no ways of prioritising patients. However, this approach is inherently inefficient because considerable effort may be required to understand each patient's medical history, e.g. examination of the patient's EHR or questioning the patient.

Currently, a patient may be able to complete a pre-screener form for a particular trial, and may for example answer questions about weight and height. In the case the patient is not eligible for a particular trial, the results of answered questions are not used again to check for availability for another trial.

Hence, most systems distinguish trials for which a patient is definitely ineligible from trials for which a patient is possibly ineligible, but go no further. They do not provide any means of assigning relative importance to the many trials for which the patient is possibly eligible. Furthermore, most systems define trial relevance in the very narrow sense of patient eligibility (i.e. the probability a potential patient meets all of the eligibility criteria) for a specific trial, not the more patient-centric model of the likelihood that a patient will participate fully and successfully in a trial (we call this ‘trial suitability’ or ‘relevance’) over potentially many different trials.

There is a need for a standard representation of clinical trial protocols that can be further presented in a machine interpretable form and in human readable form. This would allow the data collected when deciding the suitability of a particular trial to be used again for other trials and to recommend potentially relevant trials to a patient.

An automatic determination of patient eligibility requires that eligibility criteria are converted into a machine interpretable representation. Two possible approaches are (i) human annotation and (ii) automatic annotation using Natural Language Processing (NLP). However human annotation is laborious and even state of the art NLP algorithms do not have sufficient accuracy. Furthermore, NLP techniques often fail because sentence structure is too complex.

### SUMMARY OF THE INVENTION

The invention advances the field of computer-implemented clinical trial methods and systems through an approach that enables frictionless adoption by trial sponsors and provides the most accurate and patient-centric trial eligibility guidance. This approach maximises liquidity and trial participation rates.

The invention is a computer implemented method for determining clinical trial suitability or relevance, comprising the step of using answers to questions generated by a probabilistic, query-based, clinical trial matching system.

Optional features in an implementation of the invention include any one or more of the following:
the probabilistic, query-based, clinical trial matching system outputs a list of multiple different, matching trials in response to a patient answering the questions.
the list of multiple different, matching trials is ranked or ordered as a function of clinical trial suitability or relevance to that patient.
a structured, computer parseable representation of a clinical trial's eligibility criteria is used by the probabilistic, query-based, clinical trial matching system.
the structured, computer parseable representation is hierarchical and enables patient suitability or relevance probabilities to be extracted.
a structured grammar represents clinical trial eligibility criteria in machine interpretable and human readable form.
a hybrid human+NLP (natural language processing) system is used to generate a structured, computer parseable representation of clinical trial eligibility criteria.
a human annotator restructures clinical trial eligibility criteria until it is interpretable by the NLP system.
the method is further used to train a fully automated NLP system.
query-based search is used to solve the patient-trial matching problem.
a patient is matched to the most relevant or suitable clinical trials (e.g. most likely to participate in successfully) by asking the patient a series of questions generated by the probabilistic, query-based, clinical trial matching system.
questions are dynamically selected to maximize the effectiveness of the questions in improving the quality of the search results.
questions are generated dynamically to minimize the total number of questions.
questions are prioritized by calculating how likely a question will be answered, taking into account previous patients' behavior in relation to that question.
the system learns probability distributions that are then used to describe the probability that an unknown patient attribute will take a particular value.
one of the patient attributes is how likely a patient is to participate in a trial.
a statistical model of patient attributes is dynamically updated based on answers given by patients.
the statistical model of patient attributes is learned using data from a large population of patients.
further questions, independent of the normal question-generation sequence, are introduced and asked, for the purpose of improving the statistical model.
the statistical model of patient attributes uses information from patients' electronic health records.
the method includes the step of probabilistically modelling patient suitability or relevance to one or more trials.
the probabilistic modelling is a function of both patient suitability to the trial and trial suitability to the patient.
data provided by patients is aggregated during the trial matching process across multiple trials to optimise the design of future clinical trials.
data is automatically collected and aggregated from patient answers obtained during a probabilistic query-based trial matching process, to create a set of data for use in the design of future clinical trials.
conversion rate data is obtained, namely the number of patients who commence and/or complete a clinical trial that has been identified using the method for determining clinical trial suitability or relevance defined in any preceding claim.
future trial participation probabilities are estimated using data about the participation of patients in previous real trials.
the method comprises the further step of validating or assessing the accuracy of a patient attribute recorded in an HER.
the questions generated by the probabilistic, query-based, clinical trial matching system are automatically generated and are in compliance with the requirements of an independent review board, based on data input by a trial sponsor.
a trial sponsor uses a content management system to define the trial eligibility criteria and the content management system permits the selection of terms that have been pre-approved by an independent review board in order to reduce the extent of free-form text input by the trial sponsor.
a structured, computer parseable representation of a clinical trial's eligibility criteria is automatically generated based on the inputs captured by the content management system.
an alert is automatically sent to a patient if the answers previously given in respect of a clinical trial indicate suitability or relevance of a new clinical trial.
the clinical trial matching system automatically uses answers or other data from any of the following: electronic health records; data from physicians;
data from electronic health devices or services.
questions that users are likely to be able to answer are identified and prioritised as suitable questions to be asked by the system.
if a patient seems competent in answering medical questions, the system can prioritise asking that type of question.
as the patient answers more questions, the matching trial results are dynamically re-ranked as a more complete picture of the patient is built up.
the system assesses trial suitability by taking into account factors, such as one of more of the following factors: the patient friendliness of the trial; how invasive the medical procedures in the trials are; whether there is car parking for a patient; whether the trial involves an overnight stay; whether the trial requires abstinence from food or drink or other activities; the distance needed to travel; the nature of the interventions.
the system learns what weighting or discount or premium to apply to factors affecting trial suitability by monitoring whether or not patients go on to participate in trials.

Other aspects include the following:

Another aspect is a method for matching a user to suitable clinical trial(s), including: receiving a collection of computer parseable representations of clinical trial protocols, receiving an input search query from the patient, generating a series of queries based on the input search query, presenting the series of queries to the patient, and generating a list of results with clinical trials, in response to answers from the queries given by the patient.

The method may include any one or more of the features defined above.

Another aspect is a computer implemented system for matching a patient to clinical trial(s), the system comprising: a database storing computer parseable representation of clinical trials, a query-based search interface module configured to receive an input search query for a clinical trial by the patient, and to receive answers from the patient, a query-generation module configured to generate a series of queries based on the input search query and to present the generated queries to the patient, a processor programmed to, generate a list of results with clinical trials in response to the answers from the queries given by the patient.

The computer implemented system may include any one ore more of the features defined above.

Other key aspects are shown in FIG. 1 and include one or more of the following, alone or in combination:
Computer implemented system and method for determining clinical trial eligibility by using answers to a probabilistic, query-based, clinical trial matching process.
A structured, computer parseable representation of a clinical trial's eligibility criteria, enabling patient eligibility probabilities to be extracted from this hierarchical representation.
   A structured grammar to represent clinical trial eligibility criteria in machine interpretable and human readable form.
Computer implemented system and method of a hybrid human+NLP system to generate a structured computer parseable representation of a clinical trial and its eligibility criteria.
   A hybrid human system for generating a structured computer parseable representation of a clinical trial and its eligibility criteria in which a human annotator restructures a clinical trial until it is interpretable by a natural language processing system.
Computer implemented system and method for using the hybrid system to train a fully automated NLP system.
Computer implemented system and method for using query-based search to solve the patient-trial matching problem; computer implemented system and method in which queries can be dynamically selected to maximize the effectiveness of the questions in improving the quality of the search results.
   A method for matching a patient to the most relevant or suitable clinical trials (e.g. most likely to participate in successfully) by asking the patient a series of question(s).
   A method as above in which question(s) are generated dynamically to minimize the total number of question(s).
   A method as above in which the likely value(s) of patient attributes are used.
   A method as above in which the statistical model(s) are dynamically updated based on the answers given by patient(s).
   A method as above in which question(s) are prioritized by calculating how likely a question will be answered, wherein previous patient's behavior in relation to the question is taken into account (e.g. clicking “unknown” or “skip”).
   A method as above in which one of the patient attributes includes how likely a patient is to participate in a trial.
   A method as above wherein the statistical model(s) are dynamically updated based on the answers given by patient(s).
   A method as above in which the statistical model of patient attributes are learned using data form a large population of patients.
   A method as above wherein additional questions are introduced for the purpose of improving the statistical model(s).
Computer implemented system and method for the probabilistic, query-based matching of many patients across many trials.
   A method for matching many patients to many trials by asking the or each patient a series of question(s) and by modeling patient eligibility as a probability.
   A method as above in which the probability of eligibility is calculated by measuring trial relevance or suitability wherein trial relevance or suitability is a function of both patient suitability to the trial and trial suitability to the patient.
   A method as above in which information obtained from Electronic Health Records is used in generating the statistical model of patient attributes.
Computer implemented system and method of the search output being a relevance-ranked, patient-centric list of potential trials, using probability based eligibility analysis.
   A ranking search engine for patient clinical trial matching.
Computer implemented system and method for aggregating data provided by patients during the trial matching process across multiple trials to optimise the design of future clinical trials.
   A method as above further comprising the step of automatically collecting and aggregating data from patient answers obtained during a probabilistic query-based trial matching process, to create a set of data for use in the design of future clinical trials.
   A method as above wherein a probabilistic query-based trial matching process introduces additional questions (e.g. not generated in the normal order) for the purpose of improving the value of the aggregated data.
Computer implemented system and method for using answers to a probabilistic, query-based trial matching process in conjunction with EHR data.
Computer implemented system and method for obtaining conversion rate data using a probabilistic, query-based patient-trial matching system.
Computer implemented system and method for estimating trial participation probabilities using data about the participation of patients in real trials.
Computer implemented system and method for aggregating data across a population of patients to generate a statistical patient model.
Computer implemented system and method for using answers to a probabilistic, query-based trial matching process for validating or assessing the accuracy of a patient attribute recorded in an EHR.
Computer implemented system and method for pre-approving by an independent review board a structure for a trial protocol such that the trial protocol can be automatically published following any subsequent edit/update of the trial protocol (without having to be approved again).

### BRIEF DESCRIPTION OF THE DRAWINGS

The above and other aspects of the invention will now be described, by way of example only, with reference to the following Figures, in which:

FIG. 1 shows a diagram showing the different stakeholders and main components of the present invention and annotated with the key innovations.

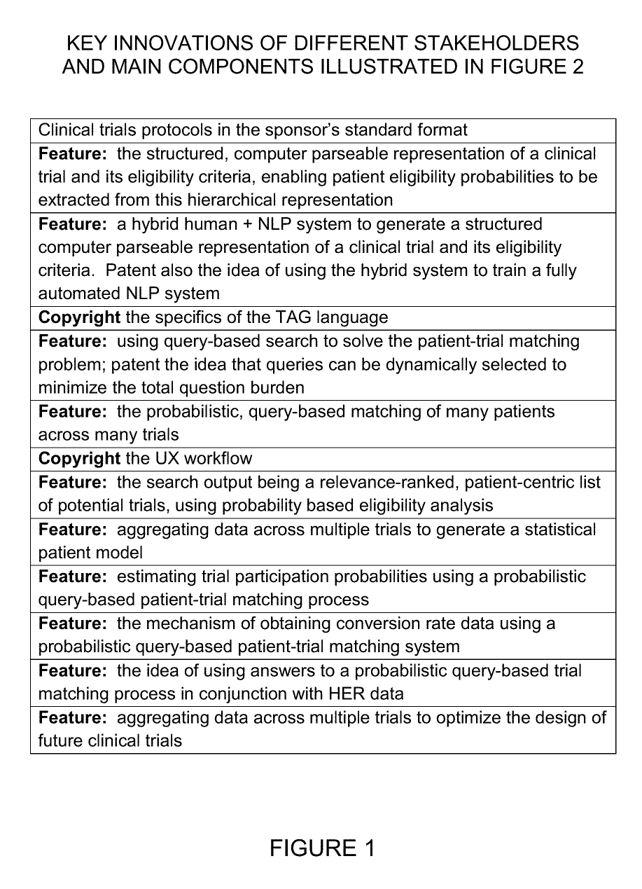

FIG. 2 shows a diagram showing the different stakeholders and main components of the presented invention.

FIG. 3 shows a screenshot of the BRIDGE content management tool.

FIG. 4 shows a screenshot of BRIDGE.

FIG. 5 shows a screenshot of BRIDGE.

FIG. 6 shows a screenshot of BRIDGE.

FIG. 7 shows a screenshot of BRIDGE.

FIG. 8 shows a screenshot of BRIDGE.

FIG. 9 shows a screenshot of a clinical trial protocol as published on a study page.

FIG. 10 shows a screenshot of a clinical trial protocol as published on a study page.

FIG. 11 shows a screenshot of a clinical trial protocol as published on a study page.

FIG. 12 shows a screenshot of a clinical trial protocol as published on a study page.

FIG. 13 shows a screenshot of a clinical trial protocol as published on a study page.

FIG. 14 shows a screenshot of the annotation editor interface.

FIG. 15 shows a screenshot of the annotation editor interface.

FIG. 16 shows a screenshot of the annotation editor interface.

FIG. 17 shows a screenshot of the annotation editor interface.

FIG. 18 shows a screenshot of the annotation editor interface.

FIG. 19 shows a screenshot of the annotation editor interface.

FIG. 20 shows a screenshot of a patient-facing web UI in which the patient can enter a condition for which a trial is sought.

FIG. 21 shows a screenshot of a patient-facing web UI in which the patient is asked to answer a question.

FIG. 22 shows a screenshot of a patient-facing web UI in which the patient is asked to answer a question.

FIG. 23 shows a screenshot of a patient-facing web UI in which the patient is asked to answer a question.

FIG. 24 shows a screenshot of a patient-facing web UI in which the patient is asked to answer a question.

FIG. 25 shows a screenshot of a patient-facing web UI in which the patient is asked to answer a question.

FIG. 26 shows a screenshot of a patient-facing web UI with a result page displaying potential eligible trials for the patient.

FIG. 27 shows a screenshot of a clinical trial protocol as published on a study page.

FIG. 28 shows a screenshot of a patient-facing web UI with a result page displaying potential eligible trials for the patient.

FIG. 29 shows a dashboard allowing one to view and analyse continuously harvested data.

FIG. 30 shows a dashboard allowing one to view and analyse the continuously harvested data.

FIG. 31 shows a dashboard allowing one to view and analyse the continuously harvested data.

FIG. 32 shows a dashboard allowing one to view and analyse the continuously harvested data.

FIG. 33 shows a dashboard allowing one to view and analyse the continuously harvested data.

FIG. 34 shows a dashboard with key metrics relating to a particular study.

FIG. 35 shows a dashboard with key metrics relating to a particular study.

FIG. 36 shows a diagram summarising the referral management process.

### DETAILED DESCRIPTION

The invention relates to an innovative, web-based search engine intended to allow patients to find relevant clinical trials easily. This section describes one implementation of this invention. In order to create the web-based search engine, a machine interpretable representation of the eligibility criteria for a large corpus of trials is first generated. The search engine then works by asking a series of questions about the patient's medical history and personal characteristics to determine the suitability for the patient of the trials in the large corpus. Questions are generated dynamically such that previous answers will decide which question is generated next. Using a probabilistic model of trial suitability, questions are prioritized so as to maximize the expected increase in the quality of the search results. The system also makes efficient use of the patient's limited budget of enthusiasm for engagement with the search engine.

The web-based search engine provides a patient-friendly marketplace that enables patients to easily search for and identify suitable clinical trials. At the same time, organisations conducting the research or trial sponsors are given the tools to generate adequate information in order to recruit a suitable corpus of candidates for their trial.

Whilst this description focuses on clinical trials, the methods described can have a more generalized application in other areas, such as searching for and identifying financial products.

This specification describes several, important novel contributions, which may include one or more of the following:
the question of patient-trial eligibility is modeled as a probabilistic one. Whilst information about a patient's medical history may be used easily to rule out trials for which the patient is definitely ineligible, that we typically have only incomplete information about the patient makes it much harder definitively to rule in trials for which the patient is definitely eligible. This presents the question of how we should judge the relative suitability of the many trials for which the patient is only possibly eligible;
the patient-trial matching problem is cast as a query-based search, where trials are ranked according to a measure of their likely suitability for the patient. Rather than merely partitioning a set of trials into those for which the patient is definitely ineligible from those for which the patient may be eligible, our system orders search results according to a broader, more patient-centric, and practically more useful measure of the trials' suitability to the patient;
the hyperparameters of the trial suitability model are refined by optimizing the system against a metric that reflects the extent to which the search engine facilitates patient participation in trials;
a new method for generating complex search queries efficiently using a statistical model of the query space is developed;
a collaborative filtering is exploited to make predictions about patients' medical histories;
the approach to patient-trial matching is motivated by web-based document search. Here, the query takes the form of a partial model of the patient that is progressively extended as the patient supplies more information about himself;
the corpus of documents comprises clinical trial eligibility criteria for a large number of clinical trials. Document relevance is modeled as a function of the trials' suitability to the patient.

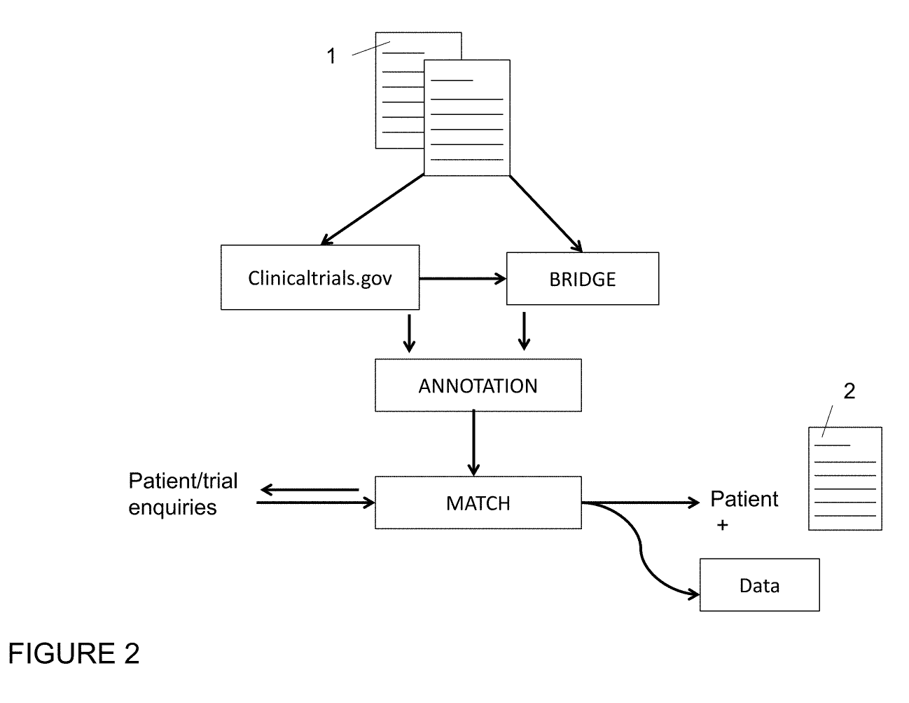

FIG. 2 illustrates the different components and process of the present invention. Clinical trial protocols are generally described in a very unstructured format (1), and are registered to clinicaltrials.gov. BRIDGE is a tool that allows clinical sponsors to edit or update information about their clinical trial. A large corpus of clinical trial protocols is edited through BRIDGE and sent through the ANNOTATION tool. ANNOTATION relates to a process of structuring plain text clinical trial protocols such as inclusion/exclusion criteria into a machine interpretable and human readable form, which is further used to power a web facing patient tool: MATCH. Anyone enquiring about a trial is able to access MATCH to interactively find suitable clinical trials. MATCH is based on a Question Based Matching System (QBMS) that processes all the available studies or trials and dynamically generates questions to help patients triage through the studies. Patients are then directed to one or more suitable clinical trials via a Study Page (2). Throughout this entire process, the entire collection of patients data across multiple trials is aggregated to further optimise the matching process and the design of future clinical trials.

Key features of this invention will be described in one of the following sections:

### Section 1: BRIDGE

### Section 2: ANNOTATION

### Section 3: MATCH

### Section 4: DATA

Section 5: Patient trial matching using Electronic Health Records
Section 6: Electronic Health Record collaboration

### Section 1: BRIDGE

BRIDGE is a web-based tool that allows clinical trial sponsors to publish their clinical trial protocol. Via BRIDGE, trial sponsors are also able to edit or/and update information for a particular trial in order to make the information about clinical trials more accessible to the patients. The structure, content and selection of terms that are available through BRIDGE have been reviewed and pre-approved by an Independent Review Board (IRB). The process of publishing trial protocols through BRIDGE therefore becomes efficient and frictionless as updated clinical trial protocols may be published automatically without the need to be approved again by an IRB.

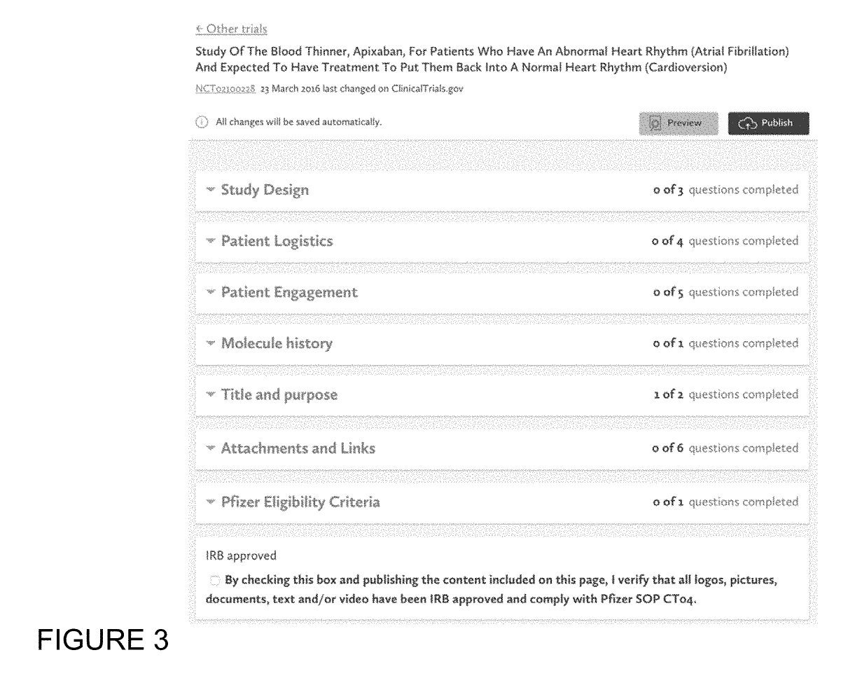

Trial sponsors may update a clinical trial protocol description as directly obtained from clinical trial databases such as clinicaltrial.gov in order to make a protocol more patient friendly. A trial sponsor may first log into BRIDGE and may find a specific clinical trial by entering the trial's NCT or EudraCT number. FIG. 3 shows a screenshot of BRIDGE related to a clinical trial with the different fields of the clinical trial organised in multiple different sections.

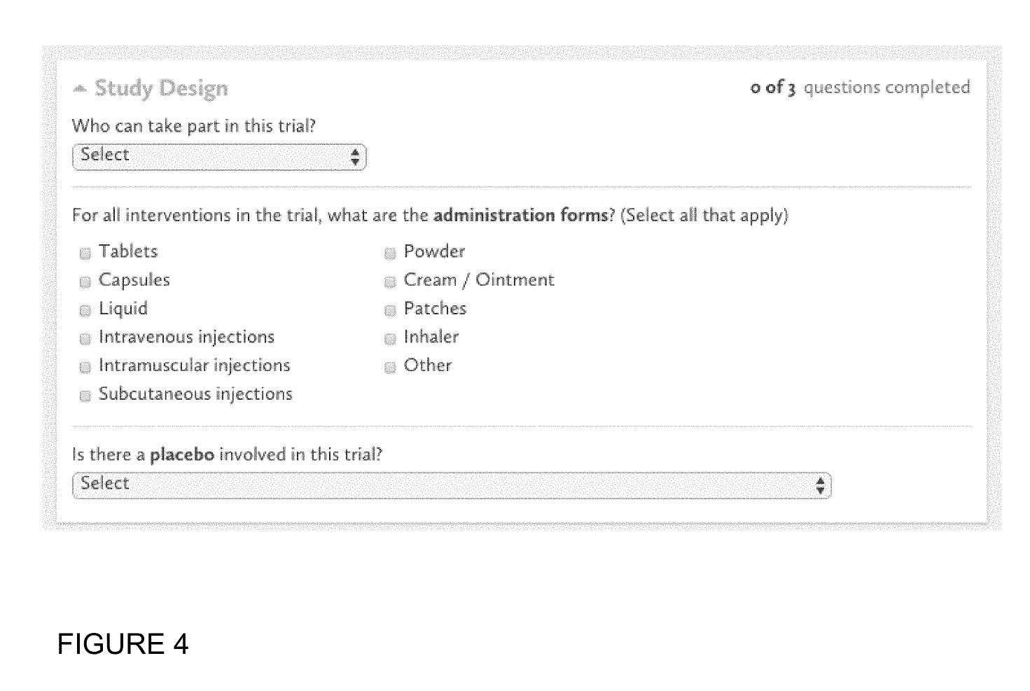

The trial sponsor may be able to edit the different fields of its clinical trial. Each field may be optional and any unanswered field may not appear on a published study page. FIG. 4 shows a screenshot of BRIDGE where the trial sponsor may edit information related to the study design of its clinical trial. The trial sponsor may select who can take part in the trial, what are the administration forms for all interventions, and if there is a placebo involved in the trial.

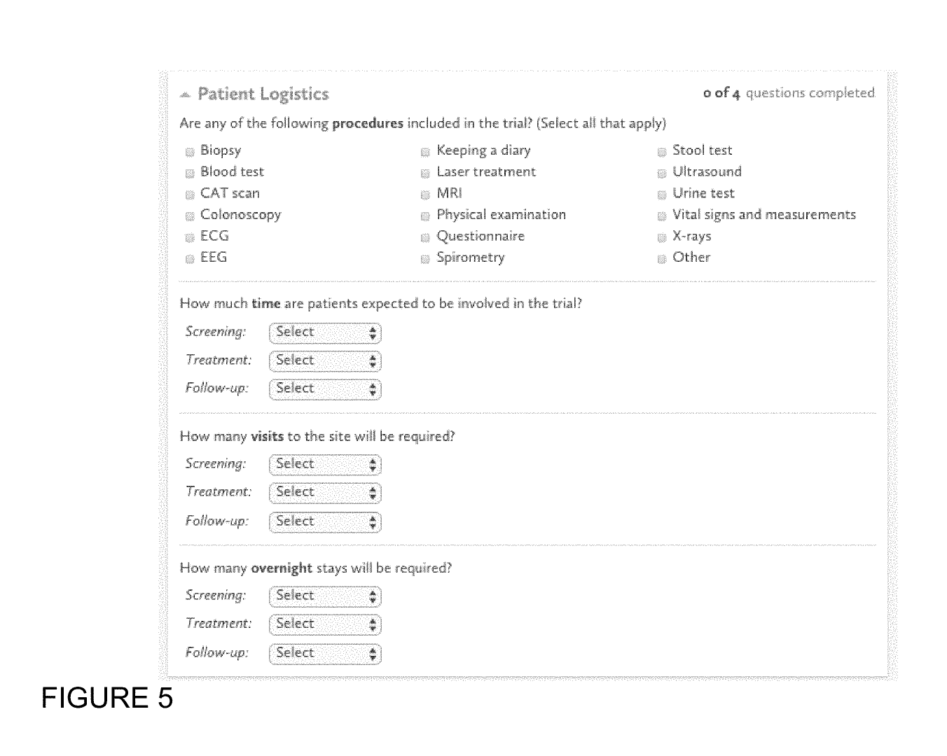

FIG. 5 shows a screenshot of BRIDGE where the trial sponsor may edit information related to patient logistics. The trial sponsor may select the procedures involved in the trial. The trial sponsor may also select information specifically related to screening, treatment and follow up, such as how much time the patients are expected to be involved in the trials, how many visits to the site will be required, and how many overnight stays will be required.

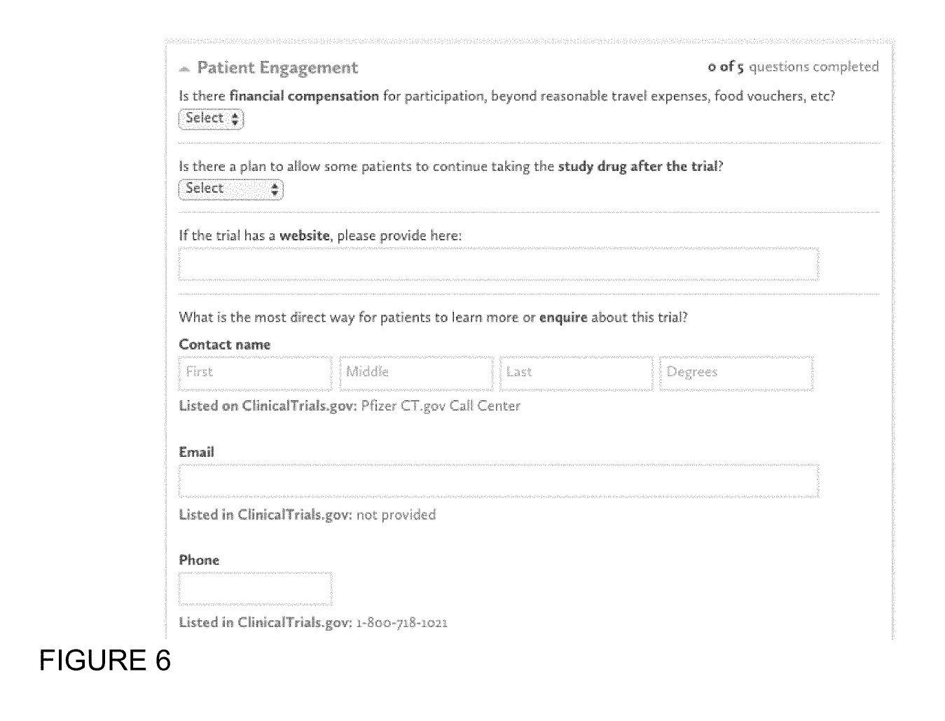

FIG. 6 shows a screenshot of BRIDGE where the trial sponsor may edit information related to the patient engagement. The trial sponsor may select information related to financial compensation and any study drug that would be available after the clinical trial has been completed. Additional information, such as a website URL or contact information may also be entered.

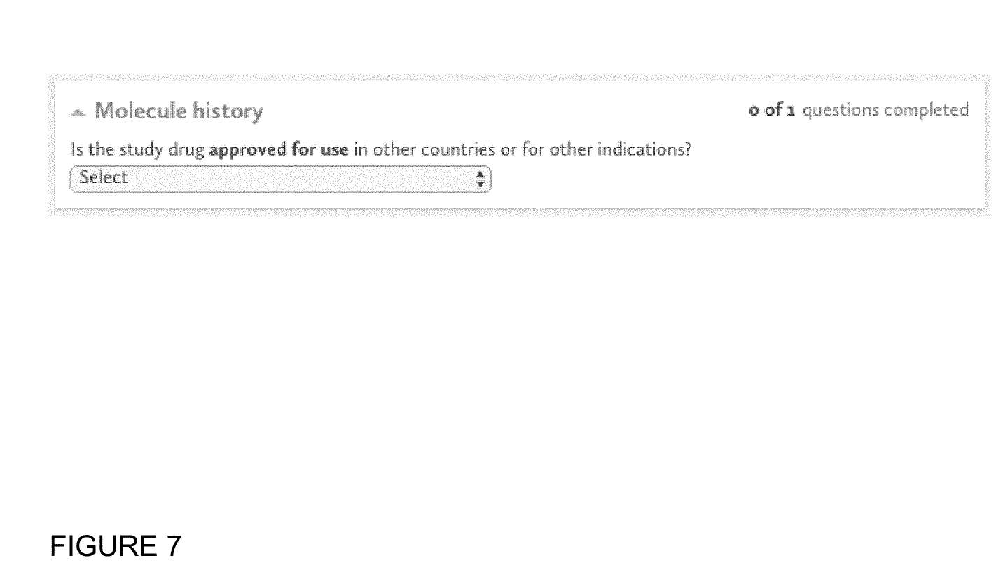

FIG. 7 shows a screenshot of BRIDGE where the trial sponsor may edit information related to molecule history. The trial sponsor may select for example whether the study drug has been approved for use in other countries or for other indications.

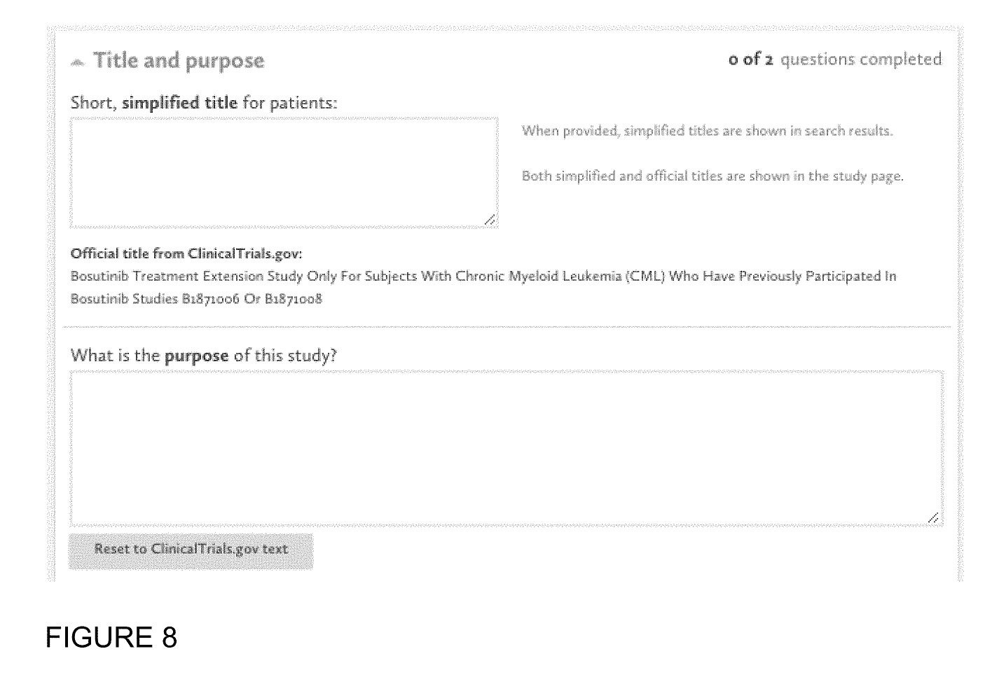

FIG. 8 shows a screenshot of BRIDGE where the trial sponsor may enter in free text a title and purpose for the study.

In addition, trial sponsors may also, for example:
add custom criteria to filter through a list of suitable patients. For example, ‘are you willing to attend 3 study visits a week?’ as it may not have been included in the clinical trial criteria;
include information relevant to the patient for the purpose of improving patient engagement by taking into account suitability for a trial (for example: whether the patient should be accompanied by a carer, possibility to continue to take the study drug if it is effective);
update their description when they are out of date;
add additional information, such as for example missing eligibility criteria that may not have been available from clinicaltrials.gov.
upload additional attachments such as documents, website links, pictures or videos.
view and edit an annotation related to the trial (as described in the following section).

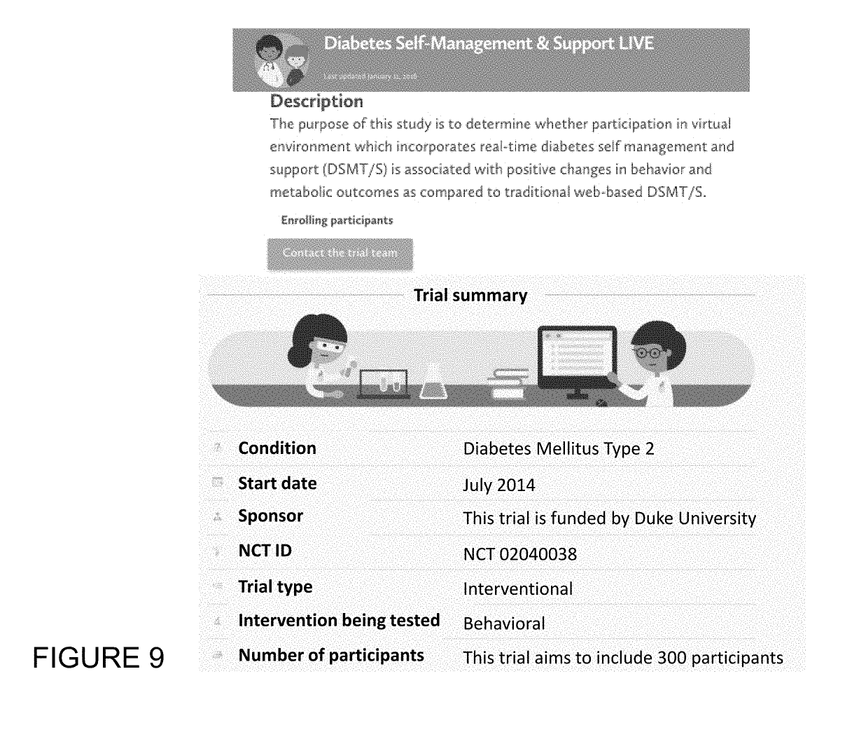

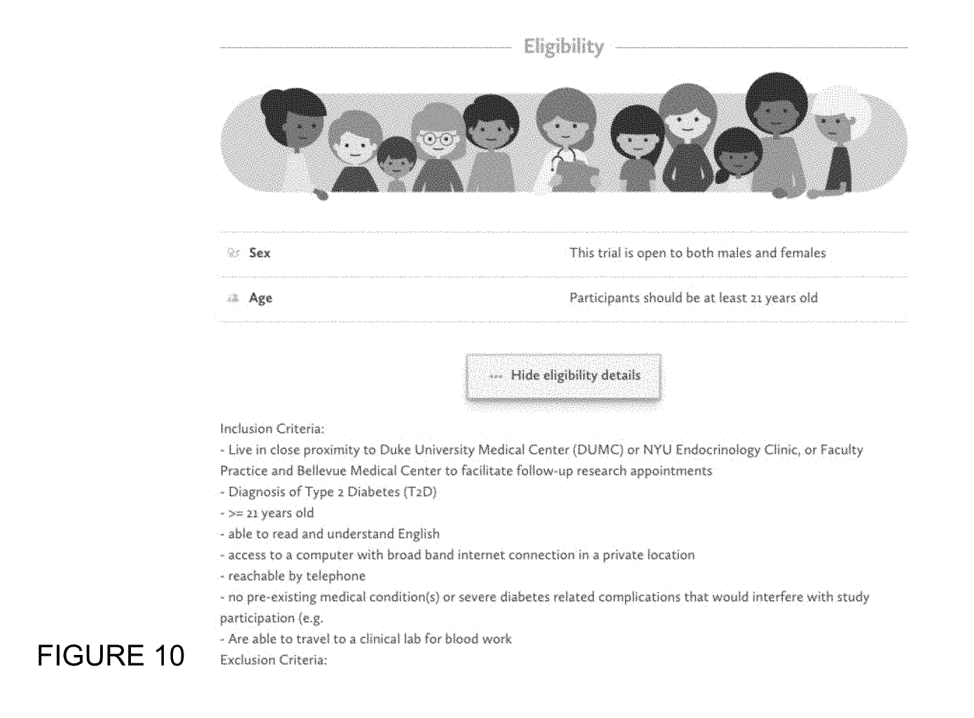

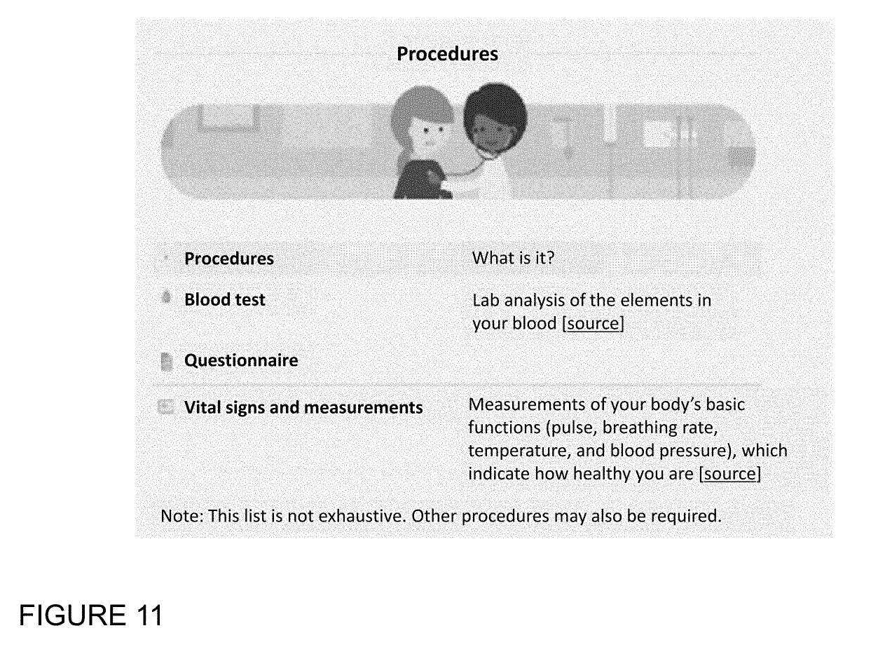

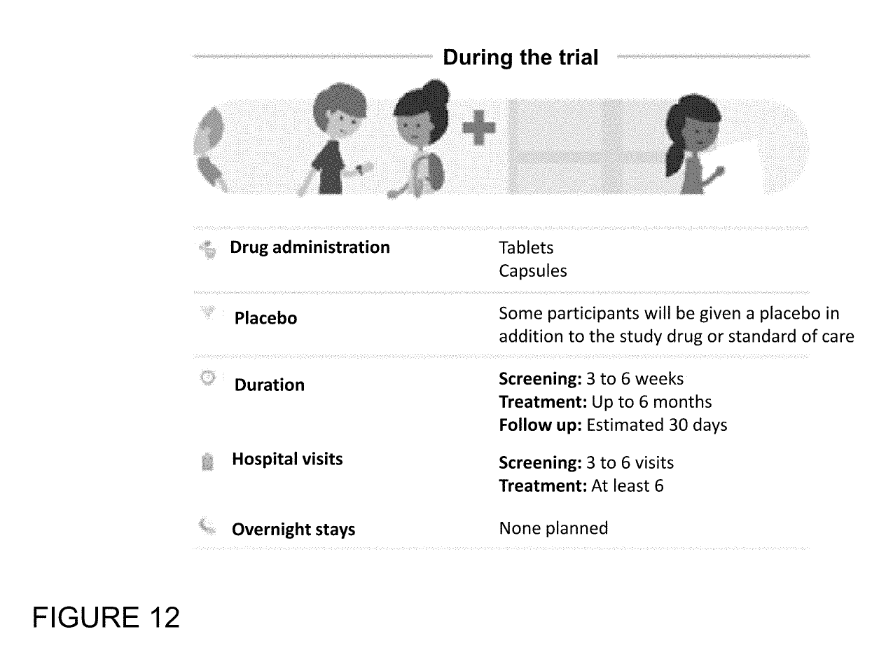

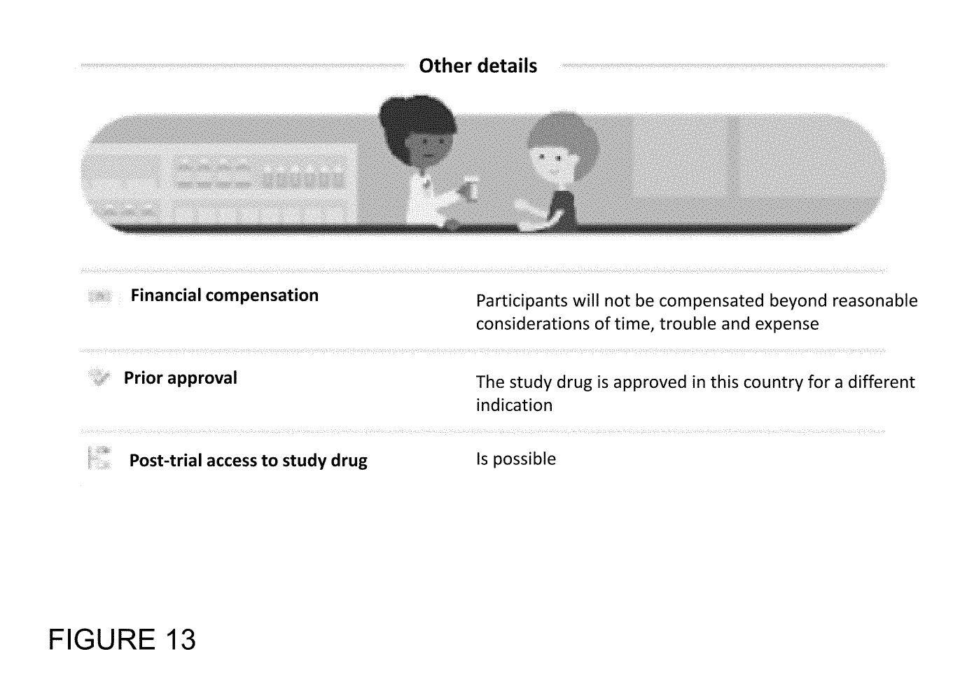

Once the trial sponsor has edited or updated a trial protocol via BRIDGE, the trial sponsor may decide to publish the trial protocol, such as by clicking on a ‘publish’ button and confirming that they are ready to proceed. The trial protocol is then published on the study page automatically. FIGS. 9 to 13 show screenshots of a study page for a clinical trial. FIG. 9 contains information such as description of the clinical trial, whether the sponsor is enrolling participants, and a summary for the trial. FIG. 10 shows a study page with a summary of eligibility with inclusion criteria and exclusion criteria. FIG. 11 shows a study page with a summary of procedures involved in the trial. FIG. 12 shows a study page with a summary of procedures involved during screening treatment and follow-up. FIG. 13 shows a study page with additional details such as financial compensation, study drug prior approval and post trial access to the study drug.

When the trial protocol is published, the original trial listing on clinicaltrials.gov is not changed in any way.

### Section 2: Annotation

TrialReach's strength is its patient-focussed partner network, and targeted machine-assisted curation of clinical trial eligibility annotation. The annotation leads to consistent and medically encoded representations of clinical trial eligibility, which are then used by MATCH as described in Section 3 and a Question Based Matching System (QBMS) to present the right next question to patients to help them triage through the studies.

By using a hybrid of two approaches, human annotation and automatic annotation using NLP, the requirement for human effort is reduced.

Hence, a hybrid system is developed which allows human annotators progressively to simplify the sentence structure of a document such as the trial sponsor's published eligibility criteria (i.e. without changing the meaning) until available NLP algorithms can accurately extract the meaning of the document. A visual feedback may also be given to the user to indicate (i) which portions of the text can be interpreted by the NLP algorithms, and (ii) what the present interpretation is. Hence, an annotator's attention can be drawn to those portions of the document that cannot yet be interpreted by NLP (so that editing efforts can be concentrated there and the annotator needs merely to check an existing interpretation, which is much faster than generating a new one).

### 2.1 Trial Annotation Grammar

A domain-specific language called TAG (Trial Annotation Grammar) has been developed to express clinical trial eligibility criteria in a machine interpretable and human readable form. TAG is used by human trial annotators to rewrite the eligibility criteria contained within plain text clinical trial descriptions.

Several important aspects have been considered when developing the structuring process. In particular:
TAG is machine interpretable and human readable.
TAG is intuitive.
TAG is simple enough to allow quick annotation.
TAG is simple to learn, (TAG can be understood by somebody with an undergraduate level of education after 3 hours of training such that they can annotate trials from clinicaltrials.gov to an acceptable level to be included in the TrialReach MATCH product).
TAG is expressive enough to cover all forms of eligibility criteria.
TAG is flexible enough to describe complex logical and temporal criteria.
TAG is able to mirror the underlying English language. (As an example, if patients must not have A or B, it might not be obvious for less experienced annotators to represent the criteria as NOT A AND NOT B. A common mistake for annotators is to write NOT A OR NOT B. TAG corresponding keyword for ‘not A or B’ is NOTANY.)
TAG minimizes mistakes in annotation from less experienced annotators.
TAG improves the effectiveness of annotators because it is easy and quick to type.
TAG is cost effective and enables a certain accuracy target to be met as cheaply as possible.
TAG facilitates the use of an autocomplete mechanism in the annotation tool. (For example, underscore prefix is placed in front of each key word).
TAG is easy to parse.
Human annotator can re-structure the original eligibility criteria, e.g. to simplify it or correct it.

Some examples of TAG keywords are described in the following sections.

### Inclusion and Exclusion Criteria

A clinical trial is associated with a set of trial Eligibility Criteria. These may be one of two things:

1. Inclusion Criteria: requirements which an applicant must have, do, or be in order to be accepted into the trial;

2. Exclusion Criteria: requirements which an applicant must not have, do, or be in order to be accepted into a trial.

All trials may have at least one Inclusion Criteria and most trials have at least one Exclusion Criteria. However, for trial annotations, all trials should have both an Inclusion Criteria and Exclusion Criteria tag. They may be represented as follows:

_inclusion_criteria
_exclusion_criteria

Inclusion Criteria tags may be added automatically. However, exclusion criteria tags may not be added automatically. Clinical trials tend to provide a header when exclusion criteria are being discussed. An example annotation may look like this:

_criterion( Exclusion Criteria: )
_exclusion_criteria

### CLAUSES

Each criterion, Inclusion or Exclusion, can be broken down into a number of propositions, such as “the patient is at least 18 years of age” or “the patient must not have cancer”. Each proposition may be seen as a question for an applicant, to which the only answers may be “yes” or “no”. The Trial Annotation Grammar is a way to logically describe these propositions in a way that a computer system can interpret and manipulate. Each proposition in the original trial criteria is represented by a Clause.

Eligibility criteria are divided into independent atoms, i.e pieces of text that can be interpreted in relative isolation from other pieces of text and which can therefore be annotated separately. One of the key benefits is the possibility of using a standard software support model for annotation, i.e. one where only hard-to-annotate independent clauses are escalated to more expensive annotators.

Table 1 provides examples of Atomic Clauses. Atomic Clauses are nouns of the trial annotation and may be categorised in four main groups:
Medical issue: _disease, _injury, _condition;
Patient attribute: _patient, _finding, _activity;
Clinical response: _procedure, _drug, _device, _treatment;
Other trial requirements: _agreement, _clinical_trial.

Each Atomic Clause is a proposition: it generally has a subject (usually the patient or candidate), and a preposition (“has disease X” or “is stage Y”). They state facts about an acceptable candidate.

TABLE 1
Example of Atomic Clauses

| Atomic | | |
|---|---|---|
| Clause | Subject | Preposition |
| _disease | A pathological process: a disease, disorder or other dysfunction | has |
| _condition | General category of something the patient “has”. This most | has |
| | commonly includes allergies, contraindications to substances, | |
| | or hypersensitivity | |
| _injury | Traumatic injury | has |
| _finding | A sign or symptom, lab or test result, mutation or histology | has |
| _patient | An attribute of the patient, such as height or weight. This can | is |
| | describe non-pathological processes they may be undergoing | |
| | (e.g. pregnancy). It can also be used for patient observations, | |
| | such as “clinical stability”. | |
| _procedure | A non-drug treatment; a therapeutic process. Includes items | has |
| | like surgery and non-surgical diagnostic processes (e.g. CAT | |
| | scans, MRI) | |
| _drug | Includes pharmaceuticals, chemotherapy, vaccinations. | takes |
| _device | An implanted or permanently attached device (e.g. insulin | has |
| | pump) | |
| _clinical_trial | An actual trial, investigative/experimental procedure or drug | has |
| _treatment | General category of treatments that do not fit well in previously | has |
| | mentioned classes | |
| _agreement | Something a candidate must have or do, such as follow an | does or |
| | exercise or dietary regimen, have access to the internet, have a | has |
| | full time carer. Most commonly, this is used to describe | |
| | “informed consent”. | |
| _activity | Things that the candidate does, often recreational, that are of | does |
| | note to the trial. This can include: drinking, smoking, exercise, | |
| | drug abuse and diet. Activities are not primarily medical in | |
| | nature. | |
| _unknown | Something that the grammar (or the annotator) can't describe. | — |
| | Use the _note keyword to explain why. | |

### Special Clause-Like Keywords

Table 2 provides examples of special Clause-like keywords.

TABLE 2
Example of special Clause-like keywords.

| | |
|---|---|
| _criterion | The original criterion text from the trial description. |
| _note | An important note regarding the annotation of the trial. |
| _meta | Automatically generated metadata. |

A _note tag may be present when a difficulty is encountered, and serves to clarify the annotator's reasoning. If the problem is self explanatory, an _unknown tag on its own may suffice.

A _note tag is not part of the logical structure of the trial and the text it contains will not probably be taken from the original criterion. An _unknown tag contains text from the original criterion and as such is a placeholder for a future annotation when the problem is resolved (eg. ‘something confusing’ is determined to be a _finding instead of a _patient or an _injury instead of a _disease, etc).

### Comparisons

This relates to things that cannot be described as simple facts. For example, a patient can either have a disease, or not have a disease. However, things like “height” or “age” may take a range of values. These things are defined as comparisons or inequalities: simple mathematical functions which evaluate to either true or false. Comparisons take the form of Comparable Operator (Threshold). There are five different kinds of comparison, or Operator:
=exactly equals
<strictly less than
<=less than, or equal
>strictly greater than
>=greater than, or equal

A Threshold is some value that the Comparable must be compared to.

Wherever possible, threshold must include units. For example, candidate ages must be in weeks, months or years, and blood chemical test results are usually in the form of milligrams, micrograms or nanograms of substance per unit volume of blood (usually decilitres or litres).

Some thresholds are relative values, such as “normal limit” or (more unhelpfully) “within reasonable limits”. In this case, the descriptive text may be inserted in the threshold position as units may not be necessary (an example is given below).

Other comparable items might not have a unit at all. Patient conditions might just be described as “stable”, patient sexes are “male” or “female”, and so on. Again, in this case the desired value may be inserted as plain text as units may not be necessary.

Patient attributes are one example of a Comparable thing. If a criteria indicates that a candidate must be at least 18 years old, the annotation may be:
_patient (age)>=(18 years).

A number of common patient comparables may exist, for example: age, height, weight, BMI, ethnicity, location, sex and life expectancy. These examples have already all appeared in many different trials.

If a trial requires a patient to have a specific location, the annotation may be:
_patient (location)=(New York City).

Lab tests are also associated with some threshold, and an acceptable candidate may have a result that must be above or below that threshold.

_finding ( serum bilirubin ) > ( 2 * the upper limit of normal ).
_finding ( fasting glucose ) < ( 100 mg/dL ).

Some lab tests may be associated with a value over a specific time period, and can be combined with a _per qualifier (see below). _per qualifiers may only relate to time periods:
_finding (eGFR)<(50 ml) _per (minute).

### Modifiers

Modifiers may be applied to an Atomic Clause in order to express some more detailed requirement.

Table 3 lists three kinds of Modifiers:

TABLE 3
Example of Modifiers.

| | | |
|---|---|---|
| negation | _no | Appears before an Atomic Clause, changing its meaning from |
| | | “patient must have/be/do” to “patient must not have/be/do”. For |
| | | example: _no_disease (diabetes) = patient must not have |
| | | diabetes. |
| temporal | _past | Appears before an Atomic Clause, changing its meaning to |
| prefix | | “history of” or “prior”. For example: _past_disease (cancer) = |
| | | patient had cancer at some point in the past. |
| | _future | Appears before an Atomic Clause, changing its meaning to |
| | | “planned” or “possible”. For example: |
| | | _future_patient(pregnant) = patient may consider becoming |
| | | pregnant in the future. |

Modifiers may also be combined together as necessary. For example:
_no_past_drug(insulin)=patient has never taken insulin

### Temporal Qualifiers

Clauses may also be restricted to mean something that only happened/happens within a certain period of time, or perhaps before/after a certain event. These are called Temporal Qualifiers.

A Temporal Qualifier has 4 main components: Anchors, Events, Operations, and Durations.

An Anchor is a point in time referencing the parent clause. Currently we support _started and _ended anchors, which refer to the start and end of the thing described in the parent clause. Anchors are optional.

An Event is a specific occasion to which a date or time could be associated. The most common event is “the start of the trial”, but there are many other possibilities. Some examples include: when a disease was diagnosed, at screening visit, or when future surgery is scheduled. Events can also be something that covers some span of time, such as “the trial”. Events are written in free text and do not have any restrictions on what an event could be.

A Duration is a span of time, including a count and some units (e.g. 1 second, 50 years).

Operators such as < and > may be inserted as necessary to describe durations like “at least 4 weeks” (>=4 weeks) and “no more than one month” (<=1 month).

An Operation associates anchors and durations, creating a useful description of a point and period in time. A list of various combinations, along with an example of the sort of thing they describe, is shown in Table 4.

TABLE 4
Examples of Temporal Qualifiers combinations.

| | | | | |
|---|---|---|---|---|
| _started | Event | | | _started (date) |
| _ended | Event | | | _ended (date) |
| _before | Event | | | _before (start of trial) |
| _after | Event | | | _after (final dose of drug) |
| _from | Event | | | _from (start of trial) |
| _from | _after | Event | | _from _before (start of trial) |
| _from | _after | Event | | _from _after (final dose of drug) |
| _from | Duration | _before | Event | _from (6 weeks) _before (start of trial) |
| _from | Duration | _after | Event | _from (6 weeks) _after (start of trial) |
| _until | Event | | | _until (end of trial) |
| _until | _before | Event | | _until _before (end of trial) |
| _until | _after | Event | | _until _after (end of trial) |
| _until | Duration | _before | Event | _until (6 weeks) _before (start of trial) |
| _until | Duration | _after | Event | _until (6 weeks) _after (start of trial) |
| _for | Duration | | | _for (3 months) |
| _for | Duration | _from | ... | _for (3 months) _from (start of trial) |
| _for | Duration | _before | Event | _for (4 weeks) _before (screening visit) |
| _for | Duration | _after | Event | _for (3 weeks) _after (end of trial) |
| _at | Event | | | _at (screening visit) |
| _during | Event | | | _during (trial) |

As a further example, _from and _until constructions may also be used together, such as:

_from (6 weeks) _before (start of trial) _until (6 weeks) _after (end of
trial), etc...

In Table 4, “ . . . ” after _for means that all of the normal _from possibilities may be used there. _until may also be used with _for clauses but again.

The during operation may not make sense for all kinds of event. A _during event must have some sort of duration. For example “_during (start of trial)” does not make much sense, because the start of the trial is an instant. _during specifies a complete duration, with an implicit beginning and end. It cannot be used with other temporal qualifiers.

Similarly, the _at operation only really makes sense for events which are a more like a point in time. For example, “_at (enrollment)” may be useful, however “_at (trial)” may not be useful.

Although some of these combinations may seem a bit clunky, they have the benefit that they are unambiguous and do not require any extra context in order for them to make sense. Trials often use constructions like “within 60 days of x”, but it is not always obvious whether this means “60 days before x”, “60 days after x”, or even “from 60 days before x until 60 days after x”. Not every combination is unique. For example: “During the trial” means the same thing as “from the beginning of the trial until the end of the trial”. Hence, more than one way to write a temporal qualifier may exist.

### Anchor Usage

The “_started” anchor is used to refer to the onset of a disease, the beginning of a course or drugs, or any other event or condition that is of interest.

In order to specify that a patient must have been diagnosed with diabetes within the last five years, the annotation may be:
_disease (diabetes) _started _from(5 years) _before (start of trial)

Similarly, the “_ended” tag refers to the end of that event or condition. The absence of a “_started” or “_ended” tag simply means that the event or condition must have been happening in the specified time period, but it does not matter if it started or ended outside of that time period.

_per qualifier for _for clauses may also be added in order to define durations of an event within a timespan:
_activity(exercise) _for (100 minutes) _per (week)

### Events

Clinical trials tend to use similar events within their eligibility criteria. Table 5 lists some examples of those common events.

TABLE 5
Examples of common events

| | |
|---|---|
| start of trial | In the absence of any other event mentioned in the trial criteria, |
| | assume that this one is meant. Its exact meaning is left deliberately |
| | vague . . . it could mean application, or screening visit, or acceptance |
| | and beginning of actual trial procedures. |
| end of trial | After the end of all trial-related activities, including surgery, drug |
| | administration, lab tests and follow-up visits, etc. |
| screening visit | A pre-acceptance test given to candidates who appear to be a good |
| | fit for a trial but may need lab tests or interviews with trial staff or |
| | medical professionals, etc. |
| visit (number) | Meetings between the candidate/patient and trial staff or medical |
| | professionals. Often appears in trial criteria as “Visit 1” or “V1”. |
| enrollment | This is another term to describe screening. After enrollment, when a |
| | patient is “enrolled”, they are in the trial. When in doubt, rely on |
| | “screening visit” or “start of trial” or annotate exactly what is in the |
| | criteria. |
| randomization | This is another term to describe the start of the trial. When in doubt, |
| | rely on “start of trial” or annotate exactly what is in the criteria. |
| | This is often assumed to mean “after enrollment but before Visit 1”. |

### For vs From

“_for” is used to specify a length of time in over which something must be continuously occurring.

“_from” is used to specify a length of time in which something must occur, but it needn't be active during that entire length of time.

For example:
_drug (metformin) _for (6 months) _before (start of trial)

The use of “_for” here means that the candidate must have been continuously taking metformin throughout the whole 6 months before the trial. It does not matter if they have been taking metformin for longer than this period of time.

The previous example can be compared with the following:
_drug (metformin) _from (6 months) before (start of trial)

The use of “_drug” here means that the candidate must be currently taking metformin, and “_from” requires that they have started metformin at some point in the last 6 months. They might have started last week or a month or six months ago, but so long as they did not start taking the drug more than 6 months ago, they will pass this requirement.

“_for” can also be used in order to specify one timespan for an event that must occur within a larger timespan. For example, the following plain text: “Have used insulin for diabetic control for more than 6 consecutive days within 1 year prior to screening”; may be annotated using “_for” like this:
_drug (insulin) _for (6 consecutive days) _from (1 year) _before (screening)

Comparison operators may also be used in_for clauses, like this:
_activity (exercise) _for <(100 minutes) _per (week)

### Complex Clauses

Clauses may be linked together to form more complex structures containing lists, possibilities, exceptions and additional details. Collectively, these things are all called Complex Clauses.

### If/Then Statements

“if/then statements” relate one complex clause with another: if the first clause is true, then the second clause can be considered. If the first clause is not true, then the second one can be ignored (won't be used to consider whether an applicant is (un)suitable for a trial).

For example, female applicants are often required to use contraception when they are involved in drug trials, but this does not always apply to male applicants.

_if _patient (sex) = ( female )
_then_agreement ( use a reliable method of contraception)

### Clause Lists

Lists of clauses can take two forms: “and lists” and “or lists”. With “and lists”, all the clauses contained within them must be true for the complex clause as a whole to be considered true. With “or lists”, if any of the clauses in the list are true, the whole complex clause is considered true.

Example: “Either insulin or metformin use” may be annotated as:

```
{
    _drug ( insulin )
    _or
    _drug ( metformin )
}
```

or alternatively,

```
_any
{
    _drug ( insulin )
    _drug ( metformin )
}.
```

Example: “All liver aminotransferase Levels no more than 3*normal limits” may be annotated as:

```
{
      _finding ( AST ) < ( 3 * upper limit of normal )
      _and
      _finding ( ALT ) < ( 3 * upper limit of normal )
}
```

or alternatively,

```
_all
{
      _finding ( AST ) < ( 3 * upper limit of normal )
      _finding ( ALT ) < ( 3 * upper limit of normal )
}.
```

Lists may not only contain items of the same type, but merely a collection of things in order to ask the question: “are all of these true?” or “are any of these true?”. Lists may also contain lists.

Example: “Known history of type 2 diabetes mellitus and glucose >110 mg/dL OR admission blood glucose ≥150 mg/dL in those w/o known diabetes mellitus”” may be annotated as:

```
{
      _disease (type 2 diabetes mellitus)
      _and
      _finding (glucose) > (110 mg/dL)
}
_or
{
      _no _disease (type 2 diabetes mellitus)
      _and
      _finding (admission blood glucose) >= (150 mg/dL)
}.
```

Lists may only contain either the very simplest kind of clauses (ones with only prefix modifiers like _no, _past and _future) or more complex clauses wrapped in braces. Anything with a Temporal Qualifier, or any kind of Complex Clause must be wrapped in braces: Example: “Have an underlying neurological disorder or suffer from a neurocognitive deficit that would affect mental status during testing” may be annotated as:

```
_disease ( underlying neurological disorder )
_or
{
      _disease ( neurocognitive deficit )
      _where _unknown ( would affect mental status during testing )
}.
```

### Exceptions

An exception to a list or general category may be made. For example: “any antidiabetic drug except metformin” or “any cancer except successfully treated cervical cancer”. This may be done by appending an Exception clause to the end of another clause, as an example:

_drug ( antidiabetic ) _except _drug ( metformin )
_disease( cancer ) _except _disease ( cervical cancer )
_where _outcome ( successfully treated ).

### Relations/Sequences

Some clauses make sense when read on their own (unlike Qualifier Clauses below) but need to be associated with another clause to give them useful meaning in trial criteria.

The most important relation clause is causation: one clause is caused by another. This is used to define things such as allergic reactions to drugs, like this:
_condition (allergy) _caused _by _drug (penicillin);

or specific kinds of treatment like this:
_disease (cancer) _treated _by _treatment (radiotherapy);

or the inverse of treated by, like this:
_treatment (radiotherapy) _treatment _for_ disease (cancer);

“by”-type and “_for”-type clauses (_caused _by, _followed _by, _treated _by and _treatment _for) can also be negated, if needs be:
_disease (diabetes) _no _treated _by _drug ( ).

### Qualifier Clauses

Additional information or restriction or requirement may also be applied to some subject other than the trial candidate. For example, the maximum dose of a certain drug that the candidate may take, or the number of occurrences of an event like a seizure.

To use Qualifier Clauses, a “_where” keyword may be attached before the any qualifier. Table 6 lists examples of qualifiers:

TABLE 6
Examples of qualifiers.

| | | |
|---|---|---|
| _dose | Of a drug, the size of the dose. | has |
| _outcome | Of a disease, surgery or drug, its result or resolution. This may | has |
| | mean successful surgery, or an unsuccessful course of | |
| | chemotherapy, or a recurrent disease. | |
| _occurrence | The number of separate occasions on which something has | has |
| | occurred, such as taking a drug or suffering a seizure. It can also | |
| | refer to more vague requirements, such as “chronic” or “frequent”. | |
| _count | The number of instances of something that happen at the same | has |
| | time has (unlike _occurrence, where they happen at different | |
| | times), such as the number of lesions found on their body, etc. It | |
| | can also refer to more vague requirements, such as “many”. | |
| _stage | Of a disease, its stage or state. | is |
| _severity | Of a disease, its grade, such as “severe” or “moderate”. | is |
| _finding | Of a disease, a specific sign or symptom. | has |
| _location | This can be used to describe as a body part or a geographic | has |
| | location. | |
| _diagnosis | Of a disease or symptom, the means by which its presence was | |
| | identified. This can be “clinical” for an official diagnosis from a | |
| | medic, “self” for diseases or symptoms reported only by the | |
| | patient. Some diseases or injuries may have specific diagnoses, | |
| | such as “radiological” for x-rays or “cytological” or | |
| | “histological” for cancer biopsies. | |

Table 7 shows some additional qualifiers for some clauses:

TABLE 7
Further examples of qualifiers.

| | |
|---|---|
| _dose ( . . . ) _per | Dosage within a specific time interval, eg. |
| (time period) | “10 mg per day” |
| _occurrence ( . . . ) _per | Occurrence within a specific time interval, eg. |
| (time period) | “>2 seizures in the last year”. |

For all qualifiers (except _outcome and_finding), you can use a comparison operator if needs be, like this:

_stage > (2)
_count < (3)
_dose > (1000 mg) _per (day)
_occurrence = (1)

The “=” comparison may not be used for these sorts of qualifiers. Here are some examples of plain text followed by the equivalent TAG annotation:

“Candidate must be taking no more than 2000 mg doses of metformin”:
_drug (metformin) _where _dose<=(2000 mg)

“Candidate is receiving doses of 10 mg or more of prednisone per day”:
_drug (prednisone) _where _dose>=(10 mg) _per (day)

“Unsuccessful surgical resection”:
_procedure (resection) _where _no _outcome (successful)

“Candidate has recurrent urinary tract infections”:

_disease ( urinary tract infection )
_where _outcome( recurrent )

“Candidate has more than three ulcers”:
_disease (ulcer) _where _count>(3)

“Candidate has stage 3 kidney disease.”
_disease (kidney disease) _where _stage(3)

Qualifiers may be combined with all of the other modified and complex clause structures. For example, for a candidate who has had more than one occurrence of severe hypoglycaemia in the 6 months before their first screening visit for the trial:

_disease (severe hypoglycemia)
_where _occurrence > (1) _from (6 months) _before (screening)

Important aspects of the grammar for trial annotation include the use of novel keywords in order to increase the representational power of the grammar.

Examples of such keywords are Subsection and Subject keywords. Trials can at times involve more than one group of patients, each with unique requirements. This is called a Subsection. Trial requirements can be directed at someone other than the patient (for example, a parent or guardian). For these, a Subject must be defined.

### Subsections

The purpose of the _subsection keyword is to distinguish criteria that relate to only one arm of a clinical trial. Criteria not included within the scope of a _subsection block are assumed to apply to all arms; criteria that are included within the scope of a _subsection block apply only to the arm named in that subsection. This allows efficient annotation of trials that have many eligibility criteria in common between several arms.

Each subsection may have an identifier (which is free text) and a block of associated simple or complex clauses. Requirements common to all subsections are left in the normal position, outside of subsection blocks, such as:

```
_subsection ( Group 1 )
{
      _patient (age) >= (18 years)
      _disease ( asthma )
}
_subsection ( Group 2 )
{
{
      _disease (COPD)
      _or
      _disease (emphysema)
      _or
      _disease (chronic bronchitis)
}
      _patient (age) <= (40 years)
}
_disease (diabetes) _from (12 months) _before (start of trial).
```

There may be one or more subsection, and each subsection may appear more than once (eg. In both the inclusion and exclusion sections).

In order to match a trial, a candidate must suit at least one of the subsections. In the example above, a candidate must have had diabetes for at least 12 months before the start of the trial regardless of age or other important illness, but must either be >18 and asthmatic, or <40 and suffering COPD (or both).

Several subsections may also have criteria in common. These may all be typed out in duplicate, or a list of subsection names may be used.

For example, a requirement may be added to Subsections A and B but no other subsections:

_subsection ( A ) _and ( B )
{
_disease ( something )
}

At times, trials may associated specific exclusion criteria to individual patient groups (or subsections) of the trial. Since only one _exclusion _criteria tag per trial may be present, _no may be placed in front of each exclusion criteria instead of using the tag. Then, at the end of the trial, a note stating that exclusion were associated with each subsection is added to the _exclusion _criteria tag, like this:
_exclusion _criteria

### Subjects

The _subject keyword is used to define eligibility criteria that apply not to the patient but to someone who has a specified relationship to the patient, such as a parent or child of the applicant.

Within a Subject block, all clauses refer to the specified subject. For example, to require that the applicant must have a parent with diabetes:

_subject ( parent )
{
_disease ( diabetes )
}.

As with the Subsection above, you can have multiple names associated with one Subject block if needs be.

_subject ( parent ) _and ( grandparent )
{
_disease ( diabetes )
}.

### 2.2 Trial Structuring

Hence the trial structuring process has several phases:

1. The plain text eligibility criteria are subdivided using standard text chunking techniques (accuracy isn't critical because the annotator can fix up chunking in the next phase).

2. A human annotator rewrites each eligibility criteria using our domain specific grammar.

3. A domain expert maps medical terms annotated in a corpus of plain text eligibility criteria onto concepts defined by standard medical ontologies.

The annotation process is built on an annotation tool that displays the annotation immediately adjacent to the original plain text eligibility criteria. The example below shows how the language is used in practice with the original content and the annotations displayed with a different color. This provides an audit trail with the benefit that annotated versions of clinical trials can be related directly to the plain text source content. Where an annotator is uncertain about the correct way to rewrite an eligibility criterion, it can be marked for later review, possibly by a more experienced annotator.

A method for computing some measure of the distance between two plain text eligibility criteria (i.e. term frequency-inverse document frequency) is developed. Criteria directly taken from the original plain text source content can be interpreted directly by doing a nearest neighbor lookup in the database criteria.

The annotation process facilitates various other machine-learning algorithms.

18 years or older
_patient (age)>=(18 years)

Glucosylated hemoglobin A1c (HbA1c) less than or equal to 12%.
_finding(HbA1c)<=(12%)

Type 1 diabetes, controlled with insulin or metformin

```
_disease( Type 1 Diabetes Mellitus )
      _and
      {
            _drug( Insulin )
            _or
            _drug( Metformin )
}
```

We have evaluated our annotation process in terms of precision (what proportion of criterion annotations are correct) and recall (what proportion of criteria can be annotated). For the diabetes diagnostic area, 95% of annotated criteria are consistent with ground truth annotations provided by a panel of three expert annotators and 95% of plain text criteria could be expressed using our grammar. To date we have structured 3,000 clinical trial descriptions obtained from www.clinicaltrials.gov using this approach.

A method for continuously monitoring changes of the source information is also developed such that updated trial protocols are sent back to an annotator to enable the annotator to make necessary modifications.

The trial structuring process makes further technical contributions, such as:
a means of drawing the human annotator's attention to patterns of annotation that correspond to common annotation mistakes has been developed. Annotation mistakes are often identified during the review phase of the annotation process—and correct and incorrect annotations provide training data that allow a machine-learning engine to learn specific features of plain text eligibility criteria and associated annotations that indicate a high probability of error. Useful features include e.g. particular syntactic constructs in the annotation and functions of the original plain text eligibility criteria, such as measures of its complexity.
a numeric unit interconversion scheme has been introduced to ensure that numerical quantities expressed in trial eligibility criteria are mapped to canonical units for use internally within the matching engine. This means that the value of a particular numeric attribute can be used to evaluate all eligibility criteria that are functions of that attribute, even if they are expressed using different units. The human annotator is warned if incorrect units appear to have been used or if units cannot be interpreted by the unit parser.
metadata labelling may be applied on an arm-by-arm basis. An additional annotation step has been introduced during which trial arms are associated with patient conditions and trial condition metadata labels. The former describes the medical condition that patients interested in the trial are likely to have, the latter describes the medical condition with which the trial is concerned. These aren't always the same, for example in a heart disease trial with an arm intended for obese patients without heart disease. These metadata labels permit a new trial filtering approach so that a subset of trials and/or trial arms can be selected using an application-specific query expression that is expressed as a function of the patient and trial condition metadata associated with each. For example, such a query expression may be used to select all trials with one or more arms intended for diabetes patients.
a simple means of associating metadata with patient attribute descriptors used in the patient-trial matching system has been introduced. This metadata enables a variety of useful functions:
   By storing the minimum and maximum plausible values for patient attributes, we can validate that the values of patient-supplied numeric-valued attributes are inside a meaningful range.
   By storing the normal range for numeric valued patient attributes, we can successfully interpret a wider range of plain text eligibility criteria for subsequent matching, e.g. criteria like “blood pressure less than upper limit of normal”.
   By storing special question wording for some patient attributes, we can override the default question generation algorithm where doing so would give a better user experience.
   By storing a flag to represent transient patient attributes we can avoid asking redundant questions of users. Transient patient attributes correspond to short duration events that are unlikely to be happening in the present, e.g. heart attack.

### 2.3 Eligibility Criteria

Clinical trial eligibility criteria define constraints on the medical history of patients who are eligible for the trial. They may be expressed as logic statements about the patient's medical history, comprised of a set of atomic logical propositions combined by the standard logic operators (not, logic-and, logic-or, if-then, etc.). By applying standard logic simplification rules, all such statements can be expressed using conjunctive normal form, i.e. as a disjunction of conjunctions (or, colloquially, a logic-or of logic-ands).

Let the logical proposition that the patient with attributes a is eligible for trial t be denoted e^{t}(a)=∈(true,false). Then patient eligibility is a disjunction of one or more conjunctions vᵢ^{t}:

e^{t}(a)=v₁^{t}(a)∪v₂^{t}(a)∪ (1)

Each conjunction vᵢ^{t} represents a seperate set of eligibility constraints cᵢⱼ^{t}, i.e.

v₁^{t}(a)=cᵢ₁^{t}(a)∩cᵢ₂^{t}₂(a)∩ (2)

and defines the set of logical propositions that must be satisfied true for a patient to be eligible for the trial.

Some additional complexity arises due to the existence of qualified eligibility criteria, i.e. criteria that express constraints on other criteria. For example, disease treated by drug, or drug given with dosage, or symptoms presented within a time period. It is important to note that qualified criteria are not the same as conjunctions of criteria (logic-ands). To see why, consider the eligibility criterion lung cancer treated by radiotherapy (expressed using our grammar as _disease(lung cancer) _treated_by_procedure(radiotherapy)). A patient who (i) has lung cancer and (ii) has received past treatment by radiotherapy would not satisfy this criterion if the radiotherapy had been used to treat a different cancer. Instead, qualified criteria give rise to symbolic references in the logic proposition, e.g. lung cancer x and x treated by radiotherapy. When attempting to determine whether a patient satisfies a qualified criteria, our system must first generate a question about the root criterion and then generates a question (or questions) about the qualifier(s). E.g. Have you had lung cancer? And (if yes), Has your lung cancer been treated by radiotherapy? In this way, both the root criterion (lung cancer) and the qualifiers (lung cancer treated by radiotherapy) may be shared between several of the trials in the corpus. It is noteworthy that the notion of qualification is not very well expressed by EMR coding schemes, and a significant benefit of our question-based matching system is that we can capture this important nuance.

The representation of eligibility criteria has been described in detail. However, the structured representation of clinical trial protocols is not only limited to eligibility criteria and can further be extended to other content provided in clinical trial protocols. As an example, medical conditions for which the trial might be relevant can also be represented using TAG, as this might also be ambiguous and not always obvious directly from the plain text of clinical trial protocols. Similarly, TAG can also be applied to represent procedures involved in the trial (i.e. not just eligibility criteria), or possible side effects that may result from the trial.

Additionally, one or more representations of the same clinical trial protocols can be generated simultaneously using TAG. Hence it is possible for example to output the following representations of the same trial protocol:
Patient friendly representation;
Physician friendly representation;
Summary representation;
Most salient criteria representation.

### 2.4 Tool to Validate Clinical Protocol Eligibility Criteria

Clinical trial protocols can contain contradictions or redundancy in eligibility criteria. These contradictions and redundancy are not always directly obvious from the way eligibility criteria are expressed. Contradictions occur when subsets of criteria cannot be satisfied simultaneously, whereas redundancies happen when criteria can be inferred from another criteria.

A system to check the eligibility criteria is developed in order to detect errors, contradictions and redundancy and to validate the eligibility criteria, resolve contradictions and remove redundancy. If all the conditions are satisfied, the system does not return any result, otherwise the system identifies the criteria that violate the conditions.

In particular, statistical models of the likelihood of (co-)occurrence of various findings, diseases, treatments, etc. are used to detect eligibility criteria that are very unlikely to be satisfiable—and therefore highlight likely bugs. Simple logical inconsistencies in answers are also used.

### 2.5 Ontology

An ontology is used to represent the domain of patient clinical trial matching. A graphical representation with nodes and edges is used to represent the domain model. The nodes of the graph represent concepts (e.g. the patient's medical conditions, treatments, activities, physical properties, times, etc.) and the edges represent the relationships between them (for example is-a-kind of is a relationship which can link the node lung cancer to the node cancer in order to represent that lung cancer is a kind of cancer).

A process has been developed to use standard available databases and update them for the application of patient clinical trial matching. For example, the UMLS (Unified Medical Language System) database is used in order to populate the ontology. This enables the ontology to stay up to date with the public domain standards. However the available standards are not always entirely suitable in the context of patient clinical trial matching. Therefore the ontology is developed in a way that it is easy to add relevant new concepts and relationships. For example many eligibility criteria may cover attributes related to patient activities and their day-to-day lives, such as for example going to the gym, dieting or running. These concepts might not always be available in the public domain and can be added to the graphical representation of the ontology with their associated synonyms and relationships.

Hence, the ontology creation process is managed like a software build process. A computer program (written in a suitable scripting language) is used to combine relevant information from many different sources into a single whole according to a well-defined and repeatable procedure. Therefore, even when one of the sources changes (for example because a new version of a public domain database is released) the ontology is quickly updated to reflect the change. Sources of information might include (i) public domain medical ontologies and glossaries, (ii) our own modifications to those ontologies (which can be modelled as software ‘patches’), and (iii) new ontologies created in the process of annotating trial eligibility criteria.

The implementation of a (semi) automatic process is also available where an annotator can decide whether (i) to map a new synonym to an existing concept or (ii) to create a new ontology concept if no existing one is a good match. For example, when a new term is encountered, an ID for the term is created and associated to a particular synonym in the model. When the same term is encountered in the future, annotations can then become automatic. A semi automatic approach for annotations can also be used where annotators are forced to make a mouse, etc. gesture to confirm the interpretation is correct.

In the annotation tool, recognised synonyms of known medical concepts may therefore be identified automatically in the input text. This reduces the amount of work to be done by the annotator, since automatically identified terms can be annotated with just a double click or other similar selection action. However, when synonyms are not recognised automatically, the human annotator can map them to an underlying medical concept ID, thereby generating a new synonym for the concept. The updated synonym table may also be shared automatically amongst multiple annotators so that all annotators can benefit immediately from updates made by one annotator.

A highlighting tool is also developed such that frequently used terms can be highlighted when they are recognised and relevant information is further displayed by looking up the ontology database. The highlighting tool can further be used to indicate that a mouse gesture etc. is needed to automatically annotate the term.

Furthermore, the ontologies enable the annotated terms to be mapped into preferred medical terminology. As an example, when an annotator types disease heart attack, the ontological relationship is able to automatically infer that the related medical term is myocardial infarction.

The ontologies are stored on a server. The server is synchronised automatically on the annotators' machine as they benefit from having the most up to date version of the ontologies. The ontologies are also accessible from the public facing tool generating questions.

### 2.6 Annotation Editor

Additionally, clinical trial sponsors may have access to an interface that allows them to write trial protocols directly such that they are structured conforming to the annotation grammar, so that it is not necessary to subsequently annotate them.

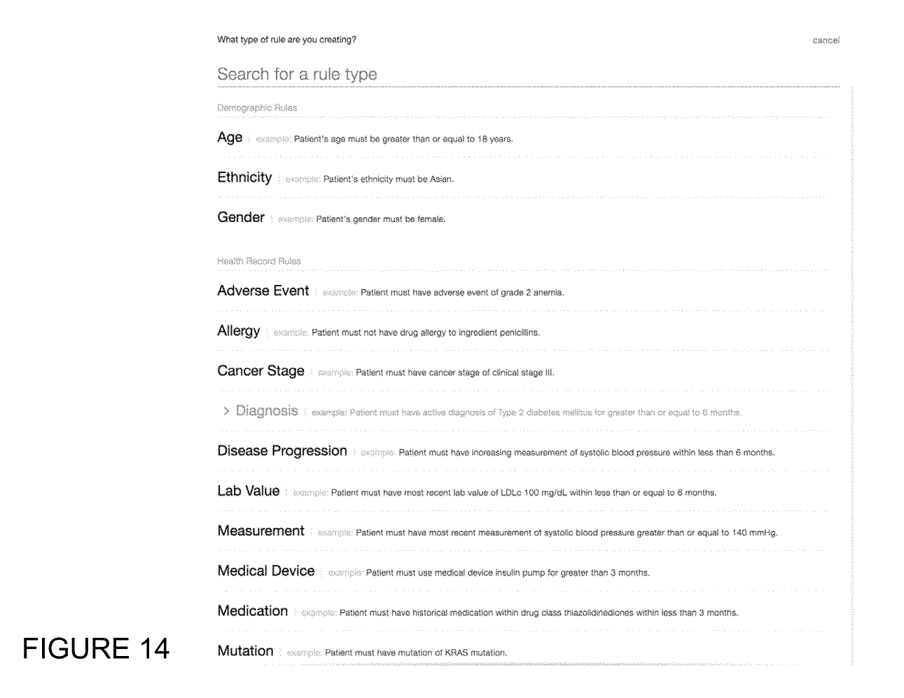

FIG. 14 illustrates an example of the annotation editor interface, which helps clinical trial sponsors to directly create structured eligibility criteria. The structured eligibility criteria may then further be automatically interpreted and manipulated by a computer system. Through the annotation editor interface, a trial sponsor is able to create a new rule or clause. The trial sponsor may search for a specific rule type or atomic clause, such as demographic rules or health record rules.

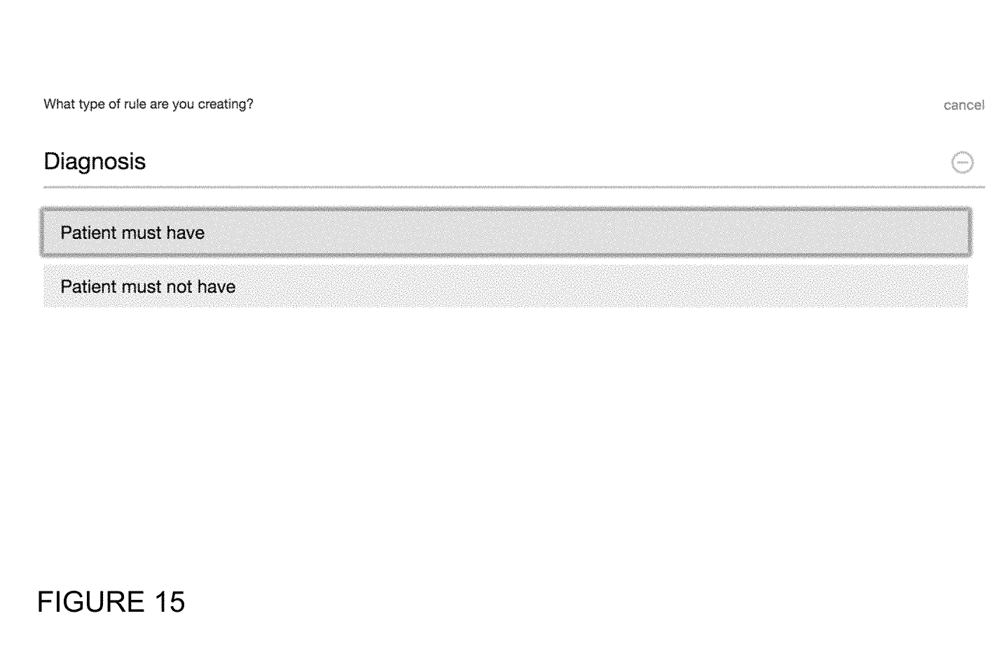

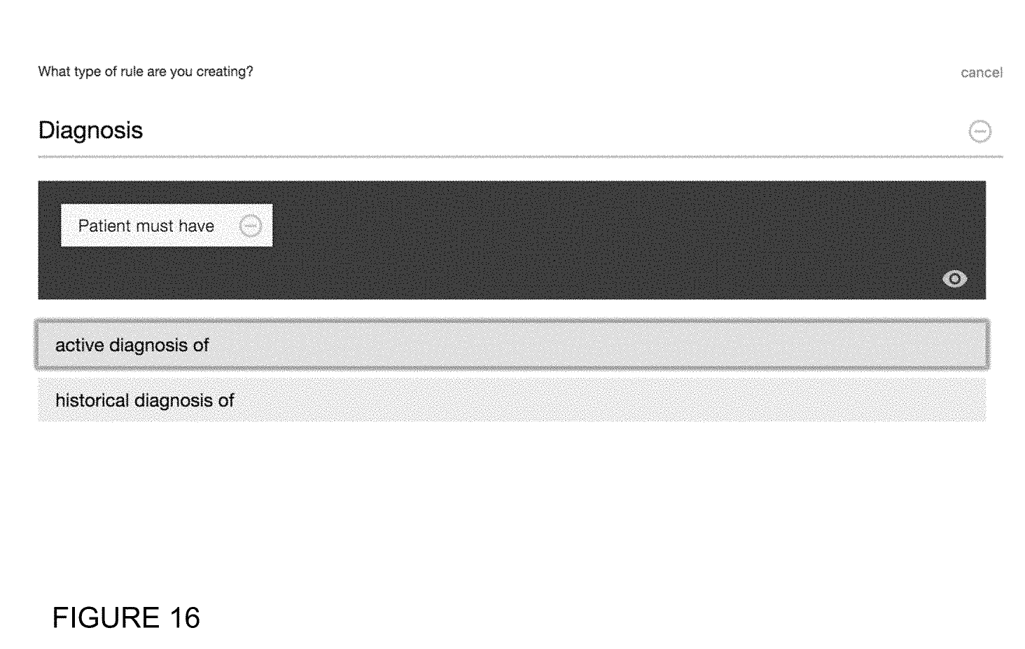

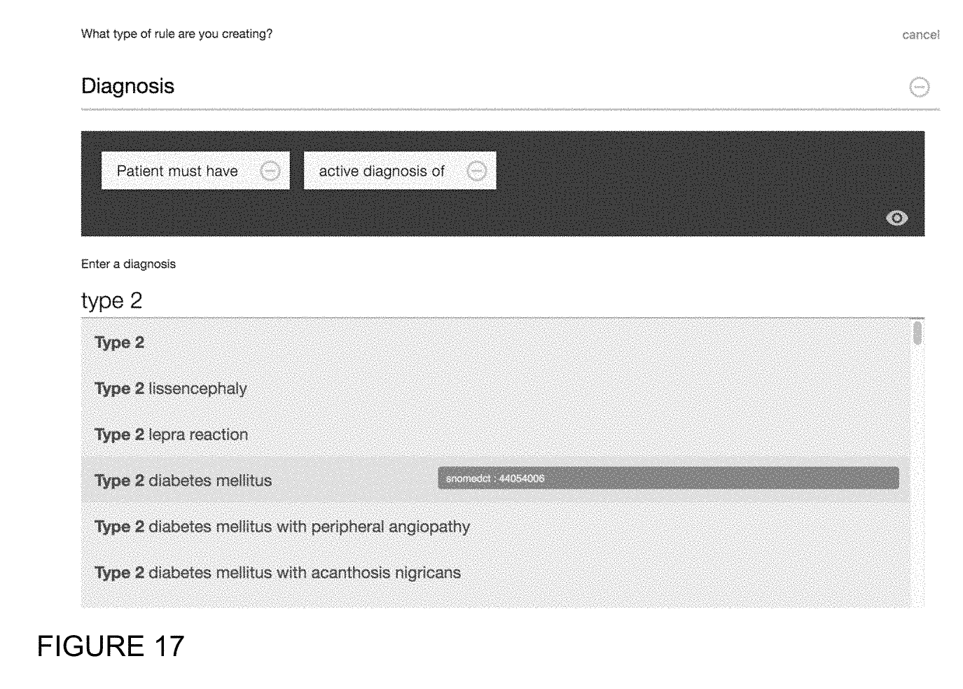

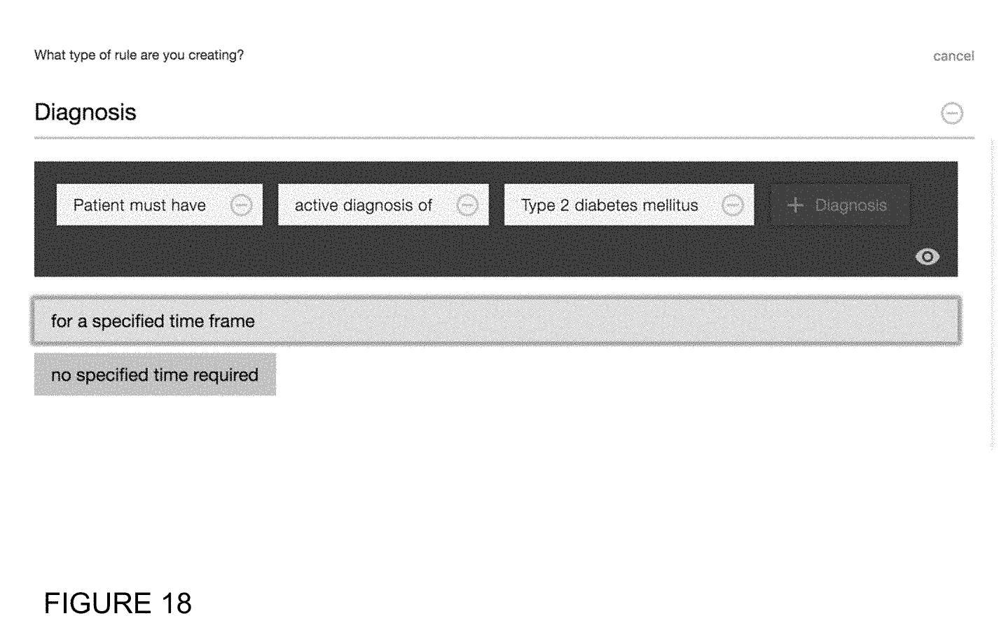

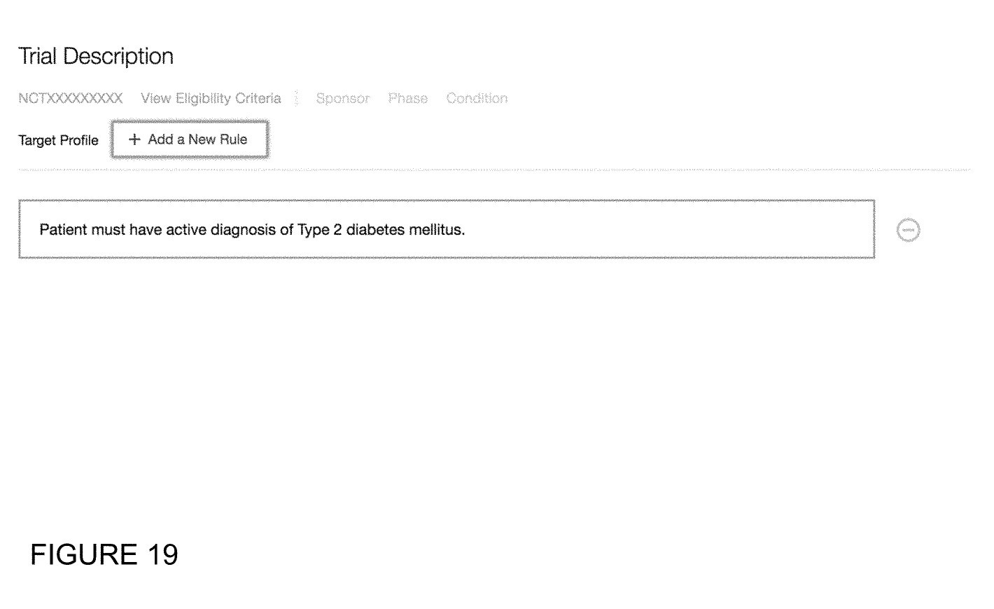

FIGS. 15 to 19 show a step-by-step example where a trial sponsor creates a new eligibility criterion specific to a diagnosis rule or clause. The annotation editor acts as a guide to help the trial sponsor creating the new diagnosis clause. The clinical trial sponsor first selects if the ‘patient must have’ or if the ‘patient must not have’ the diagnosis as seen in FIG. 15. Next, the clinical trial sponsor specifies if the new rule or clause refers to an ‘active diagnosis’ or an ‘historical diagnosis’, as seen in FIG. 16. Suggestions of diagnostic concepts from the ontologies are then automatically displayed as seen in FIG. 17. The clinical trial sponsor then specifies additional temporal qualification, as seen in FIG. 18. Finally, the rule is saved and is automatically expressed in a patient friendly text, as shown in FIG. 19. The new rule may then be expressed as a structured data such that it can be used in the question based matching system:

```
[
      {
      “description”: “Patient must have active diagnosis of Type 2
diabetes mellitus.”,
      “type”: “diagnosis”,
      “include”: true,
      “qualifier”: “active”,
      “inputs”: [
            {
                  “purl”:
            “http://purl.bioontology.org/ontology/SNOMEDCT/
            44054006”,
                  “prefLabel”: “Type 2 diabetes mellitus”,
                  “description”: “Type 2 diabetes mellitus”,
                  “system”: “snomedct”,
                  “code”: “44054006”
            }
              ]
      }
].
```

### Section 3: MATCH

Given a machine interpretable representation of the eligibility criteria for a corpus of clinical trials and some information about a patient's medical history, it would be desirable to determine automatically whether the patient is eligible for any of the trials. Typically information about the patient may be obtained by either (i) asking a series of questions via a web UI or (ii) from the patient's EHR. Unfortunately, these sources of information inevitably yield only incomplete patient data. Whilst partial information about the patient may suffice to rule out trials for which the patient is definitely ineligible (because violating even a single one of the eligibility criteria is enough to rule out the trial), it may not suffice to establish that the patient is definitely eligible for any particular trial (because this requires that all its eligibility criteria are satisfied).

In the event that a patient is neither definitely ineligible nor definitely eligible for a trial, further investigation or processing may be used to resolve the question. However, an important question concerns how we might prioritize the individual trials for further investigation or processing. Thus, a method has been developed for prioritizing trials based on a measure of the probability of patient participation, suitability, relevance, or eligibility (and, optionally, some measure of our confidence in our estimate of that probability). Such a measure would provide, for example, a principled means of ranking candidate trials in a search engine UI, prioritizing further questions about the patient's medical history, or prioritizing patients for screening lab visits, etc.

A method for producing an estimate of the probability of patient-trial eligibility is also developed by using a statistical model of patient's attributes obtained (or ‘learned’) using a data about a large population of patients. Specifically, we learn probability distributions that we can use to describe the probability that an unknown patient attribute will take a particular value.

### 3.1 Patient Attributes

State-of-the-art approaches to EMR (Electronic Medical Record) coding represent patients' medical histories as directed acyclic graphs (DAGs). The nodes of the graph represent concepts (e.g. the patient's medical conditions, treatments, activities, physical properties, times, etc.) and the edges represent the relationships between them (e.g. the patient has the disease lung cancer, which has been treated by radiotherapy).

The set of interesting patient attributes may be represented by a vector:

a=[a₁,a₂, . . . ]′ (3)

where each attribute aᵢ is defined on a (possibly infinite) set Sᵢ of possible values depending on its type and the range of values that are allowed, i.e.

aᵢ∈Sᵢ (4)

For illustration, the Boolean attribute _drug(x) is defined on {True, False}, the numeric attribute _finding(HbA1c) is defined on the range (0; 100)%, the attribute _patient(sex) is usually defined on the discrete set {Male; Female}, etc.

For a given patient, attributes may have known or unknown values. Without loss of generality, a may be partitioned as a=[g u]′ into known and unknown components, g and u. When the patient answers questions presented in the UI, the set of known attributes increases. It may also be useful to distinguish unknown attribute values (about which no question has been generated) from ‘known unknown’ attributes (which the patient has indicated that he or she does not know the attribute). In the context of dynamic question generation, it is important to keep track of questions that the patient is already known to be unable or unwilling to answer so as to avoid generating the same question again in future.

The attributes of patients that may be represented by a vector can have a number of different forms. Examples are but not limited to:
Boolean form such as True/False;
Real value (for example the value of resting heart rate);
Discrete set (for example ethnicity: Caucasian/White/Other);
Mechanism by which we acquire the patient (ex: Facebook user).

Attributes can also include the ‘knowledgeability’ of the patient or the likelihood of the patient knowing the value of an attribute. These attributes are measured for example when the patient decides to click either on a ‘skip’ or ‘I don't know’ button instead of providing an answer to a particular question. Furthermore, if a patient never answers certain questions, it is possible that the questions are worded badly or have complicated medical terms that need to be phrased differently. By providing a ‘don't know’ button or similar, the understandability weightings for ontology concepts may be learnt using data about the behaviour of real users.

Hence, an understandability (‘or patient friendliness’) weighting may be stored for each concept in the ontology concept so that generated questions may be selected so as to achieve the optimal compromise between patient friendliness and informativeness.

Accordingly, the patient friendliness can be represented by an attribute and can also be modelled. If patients tend to skip medical questions then we can dynamically prioritise the non-medical questions. A per user knowledgeability model may be dynamically modelled to determine the right weight to give to patient friendliness vs. informativeness in question generation as discussed in section 3.

Patient friendliness information or patient statistics may also be used to generate good illustrative examples of what is meant by a question, e.g. “are you taking drugs to treat type II diabetes?” (e.g. metformin, insulin).

Preferred questions that users are likely to be able to answer may be learnt (in addition to preferring questions to which the answer would be informative).

Conversely, if the patient seems competent in answering medical questions, we can prioritise that type of question.

### 3.2 Probabilistic Modelling of Patients

Logical Inference

Known attribute values may be given or inferred. A patient's answer to a question defines the value of a patient attribute. However, knowing the value of one or more patient attributes may be sufficient to allow us to infer the values of additional attributes. We exploit two types of inference: computed inference and ontology inference.

Computed inference allows us to infer attribute values that can be computed from other values. For example, Body Mass Index is computed from the patient's weight (in kg) divided by the patient's height (in m) squared. Another example of a computed attribute is drug dosage per unit body weight.

Ontology inference. Ontologies provide categories for medical terms and form a directed graph in which the nodes represent concepts such as drugs or diseases and the edges represent relationships between those concepts. For example, an ontology might classify a specific drug as a kind of a broader superclass of drugs.

Such is-a-kind-of relationships allow us to make two important kinds of inference about Boolean valued patient attributes (such as _drug(x)). Let the concept A be a superset of concept B so that ∀B⊆A

Ā→B̅ (5)

B→A (6)

The first statement means that the absence of a superclass implies the absence of the subclass. For example, the absence of cancer implies the absence of the subclass lung cancer. The second statement means that the presence of a subclass implies the presence of the superclass. For example, the presence of lung cancer implies the presence of the superclass cancer. Both of these inference rules are applied recursively, so e.g. the absence of cancer may additionally be used to infer the absence of any subclass of lung cancer. However, in each case, inference is complicated by the existence of multiple inheritance in the ontology, i.e. of concepts that are children of more than one superclass. Our system addresses the problem of multiple inheritance in ontologies by not using for inference any is-a-kind-of relationship that connects a parent to a child with more than one parent. For illustration, the drug biguanide is classified by the ICD ontology both as a kind of anti-malarial drug and as a kind of anti-hypertensive drug.

Thus the fact that a patient has taken biguanide cannot be used to infer that the patient has taken an anti-malarial drug (because the patient may have taken an anti-hypertensive biguanide). Similarly, that the patient has not taken an anti-malarial does not imply that the patient has not taken biguanide (because the patient may have taken anti-hypertensive biguanide).

Furthermore, the logical inference engine may also be extended to allow inference over constraints on patient attribute values instead of just values.

An example of inference over Boolean valued attributes may be:
_no _patient(diabetes)→_no _patient(type 2 diabetes).

This may be extended to conduct inference using more general patient attribute inequality constraints, such as:
_patient(pregnant)→_patient(age)>=10, _no _patient(female)→_no _patient(pregnant), etc.

Our constraint-based logical inference engine is implemented using a graph. The nodes of the graph represent patient attribute constraints (e.g. _patient(age)<10 or _disease(diabetes)=true) and logic operations (logic-and, logic-or, and not). The edges of the graph model logical inference. When a particular node is satisfied, nodes connected to it by an edge are satisfied too.

This extension to the logic inference engine underpins some other extensions to the system's functionality:
We can perform more nuanced detection of mutually incompatible or redundant eligibility criteria in structured representations of trials. During trial validation, the validation engine explores inference graph to expand each eligibility criterion in turn to see whether it is incompatible with any of the other criteria. For example, two eligibility criteria requiring both _disease(diabetes) and _disease(type 2 diabetes) would be redundant, because _disease(type 2 diabetes) infers_disease(diabetes). Conversely the constraints _patient(age)<2 and _patient(pregnant) are incompatible because _patient_(pregnant) implies _patient(age)>10.
Given some information about the patient, we may be able to infer tighter constraints on the range of valid patient input, thereby increasing the quality of our data by reducing the possibility of certain kinds of data entry mistake. For example, knowing that the patient is pregnant means that we should reject patient ages less than 10 as being incompatible with our existing knowledge. The previous Boolean inference engine allowed us to use given information only to rule out entire questions.

Statistical Inference

Logical inference allows us to reach logical conclusions with certainty, e.g. that a patient with type 2 diabetes certainly has (a form of) diabetes. But when we can't reach certain conclusions, we may still be able to increase our understanding of what is likely, e.g. that a patient is likely to have type 2 diabetes given that the patient has diabetes. (In the UK, a patient has a 90% chance of having type 2 diabetes given that he has some form of diabetes.) Where logic is concerned with what is certain, statistics is concerned with what is likely.

In general, the value of each unknown patient attribute aᵢ is governed by a prior probability density p(aᵢ) (or, in the case of attributes that can take a discrete set of values, by a probability distribution P(aⱼ).). Now, given some information about the patient, say attribute b has known value \hat{b}, in general the distribution of a varies to p(a|b=\hat{b}).

For example the probability density function of an unknown BMI of the patient will be updated when the patient has entered its weight, as BMI is a function of weight and height.

Statistical models are used because a level of uncertainty is always present, even after checking the electronic health record and even after every possible question has been asked. However, given the known attributes from a patient, the probability of the next answer to be true can be calculated. For example, given the patient has cancer, what is the probability the patient has been treated previously by chemotherapy?

The probabilistic models of patient attributes and eligibility also help with the prioritization of patients, for example which patients could usefully attend a screening visit, or need a follow up, or which one should go for a physician visit in order to have their electronic health record reviewed. Probabilistic models enable statistical inference of attributes (e.g. assuming those attributes follow a Gaussian distribution curve or suing Bayesian inference).

Probabilistic Eligibility

Given known patient attributes g, the probability of the random event E^{t} that a patient is eligible for a trial t is given by the expectation of eligibility over all possible values of the unknown patient attribute values:

P(E^{t}|g)=P(e^{t}=true|g)=∫ᵤe^{t}(g,u)p(u|g)du (7)

where here the integral symbol means integration for patient attributes defined on a continuous space and summation for those defined on a discrete space.

Given enough data about real patients, it would be possible to learn the family of conditional probability distributions p(u|g). In practice, it is useful to approximate the conditional distributions by assuming conditional independence:

p(u|g)=p(u₁|g)p(u₂|g)p(u₃|g) (8)

We can further approximate the model by replacing the conditional distribution with the prior for those attribute values that cannot be inferred by logical or computed inference from known attribute values, i.e.

$$p ( {u}_{i} \vert g ) = \{ \begin{matrix}δ ( {u}_{i} ( g ) ) & {u}_{i} ( g ) is known \\ p ( ui ) & otherwise\end{matrix} \qquad ( 9 )$$

where here δ(·) should be interpreted as the Dirac delta function when the patient attribute uᵢ is defined on a continuous space and the Kronecker delta when it is defined on a discrete one.

Even in the case that some patient attributes have unknown values it is possible to infer the probability that a patient will satisfy eligibility criteria for a particular clinical trial.

Therefore, for a particular trial, it is possible to discover a percentage of the population that might be suitable for a trial. This is mainly due to the fact that to be ineligible for a trial only a single criterion has to fail, whereas to be eligible for a trial all of the criteria have to be satisfied.

A prioritisation engine generates questions specifically to help populate and improve the model.

In addition, until a patient goes to the trial site and talks to the investigators of the trial for a secondary screening, the eligibility of a patient cannot be certain. Therefore, additional attributes such as what happens after matching the patient to the trial can also be modelled, for example whether the patient has satisfied the secondary screening and whether the patient went on the trial and finished the trial. These attributes can also be modelled and it is therefore possible to calculate for example the following, but not limited to:
Probability to fail secondary screening;
Probability to show up at secondary screening;
Probability to go through trial;
Probability to finish trial.

### 3.3 Question Generation

In order to create the web-based search engine, a machine interpretable representation of the eligibility criteria for a corpus of trials is first generated. As a simplified example, eligibility criteria may be assumed to be simple Boolean functions of the patient's present condition and medical history, e.g. “age greater than 17” or “does not have cancer”. Then a set of trials for which the patient is compatible are then determined by asking a series of questions, such as “how old are you?”, “do you have cancer”?. The answers to such questions can be used to filter or re-rank the list of compatible search results. Unfortunately patients have limited patience for answering questions and so it is beneficial if the questions are presented in an order likely to minimize the total number of questions asked.

If the eligibility criteria for a corpus of trials depend on a total of N unique patient attributes, then answering N questions would always be sufficient to determine eligibility for every trial. However, rather fewer questions may suffice in practice (say n, where n<<N). One reason is that failure to satisfy any of the eligibility criteria for a trial (which means the patient is definitely ineligible) means the satisfiability of its remaining eligibility criteria becomes irrelevant. Another reason is that it is sometimes possible to infer some attribute values from others using external sources of information such as ontologies.

Given a statistical model of the patient, it is possible to compute the expectation of n, E(n) over all trials, i.e. the number of additional questions we expect to have to ask to determine the patient's eligibility for all trials. Therefore, we have a method for choosing an order for the questions so as to minimize E(n) in light of successive answers. Note that the order of questions is not defined statically in advance, because every time the patient answers a question we gain more information about the patient, which affects the optimal ordering of future questions. Hence, the optimal order by exhaustive search, i.e. just computing E(n) for every possible ordering of all relevant questions and selecting the ordering that gives smallest E(n). Furthermore, statistical model may also be dynamically refined based on the answers given by the patients.

There are two approaches to understanding exactly which metric should be used to select each subsequent question:

1. Questions are prioritized so as reduce the number of trials for which the patient is definitely not eligible as quickly as possible.

2. Questions are prioritized so as to increase the expected increase in standard search engine scores such as NDCG10. This approach encourages the engine to generate a few good results towards the top of the ranking even at the expense of including more irrelevant results lower down in the ranking.

Clearly the second approach depends on being able to predict the expected relevance of each trial, which necessitates having a reasonable (if not perfect) model of patient preference. A simplistic approach might be to question the patient directly about his preferences, for example by asking how far he is willing to travel to a trial site, or how many trial sessions he is willing to participate in.

A more sophisticated approach is to learn the parameters of a parametric model of patient preference given information about the participation in trials by previous patients.

NDCG (normalized discounted cumulative gain) approach: each result in the list of trials page result gets a weight. The first result gets a higher weight than the second and so on. It might be more important to patients that the topped ranked results correspond to trials that are suitable, rather than to present the largest possible set of suitable trials.

The prioritization engine can be optimized according to either metrics above or according to a combination of them.

Questions are generated dynamically—i.e. the sequence and nature of questions asked progressively narrows down depending on earlier answers. Questions are asked that, if answered, maximally improve the quality of the results and hence minimise the total number of questions that need to be asked.

Questions can be generated in order to improve the quality of the results presented. However, many different measurements of the quality of the results presented are possible. For example, the quality of the results can be measured as the number of the questions required in order to settle the suitability of the trial as quick as possible. Hence in that case, the quality measurement is calculated after every single answer is given.

In addition, by analysing the eligibility criteria for a corpus of structured trials, it is possible to determine which criteria co-occur most frequently so that fewer questions are asked of users by combining multiple criteria into single questions (along the lines of “do you any of the following diseases?”). Patient-friendly ways of paraphrasing sets of questions may also be discovered, e.g. where a larger battery of tests is indented to show “normal liver function”.

Question Generation to Refine the Patient Model

One way of refining the statistical patient model to infer distributions over unknown patient attribute values is to obtain data from real patients. However, data provided by patients during the course of normal interactions with the question-based matching product is statistically biased since which questions are generated depends on a patient's answers to previous questions. Therefore it isn't well-suited to the purpose of learning general models of population statistics. This gives rise to an interesting innovation, which is occasionally to generate additional questions independent of the normal question-generation sequence purely for the purpose of harvesting statistics about patients. For example, additional questions might be generated with some probability by sampling at random from a list of additional questions. If the answer to the additional question can be inferred from answers to previous questions, then the question isn't presented to the user, but the inferred answer is still used to update the statistical model. Injecting some proportion of additional questions in this way can be thought of as imposing a ‘tax’ on the patient. It reduces the efficiency of the patient trial matching engine in the short term (because the additional questions aren't in general the maximally informative ones), but it provides data that will benefit all patients in the longer term—because a better statistical model results in more efficient patient-trial matching. The tax can be varied depending on the origin of the traffic to the patient-trial matching web site according to a variety of different commercial factors (such as the origin of the traffic to the service, the diagnostic area, the engagement of the user, etc.).

Generation of Compound Questions

The system may also generate compound questions, i.e. questions with several parts, each of which is answered independently with true or false or unknown. For illustration, such a question might be worded: “Do you have any of the following diseases?” followed by a list of diseases each with an associated check box (which may provide the option to answer “I don't know” as well as true or false). The advantage of asking compound questions like this is that the patient can provide more information more quickly since he or she can provide several pieces of information without reading several questions or waiting for the browser window to refresh. One complexity associated with multi-part question generation is the possibility that some of the question parts might have answers that are mutually incompatible under the system's inference rules. For example, to a compound question about which diseases the user has with answers including diabetes and type 2 diabetes, it would be invalid to answer “yes” to type 2 diabetes and “no” to diabetes. Then a difficulty is to communicate to the user why their input was invalid. One solution to this problem is to avoid generating any parts with incompatible answers by checking that no answer to any part could be used to infer any answer to any other part. An alternative is to present the user with information about why a supplied answer is invalid in a dialog box or similar. Another interesting challenge is to generate question parts that feel sufficiently closely related to make sense as belonging to the same question. This can be achieved by selecting question parts corresponding to medical concepts that are sufficiently closely related in standard medical ontologies.

### 3.4 Trial Ranking

Most existing work approaches the patient-trial matching problem from the perspective of patient eligibility: in other words, whether or not the patient meets the requirements of the trial. This approach has several important limitations.

Firstly, it is generally difficult to determine patient eligibility with absolute confidence. Then, given a large number of trials for which the patient is only possibly eligible, it is very difficult to choose between them, e.g. in order to rank a set of search results. It is well known that when presenting a list of search results, the highest ranks get more attention from the user than the lower ranked search results. It is more important that the top ranked search result is relevant than that a lower ranked search result is relevant. A ranking measure that fails to take this into account will provide a sub-optimal user experience. Secondly, even if a patient is eligible for a trial does not mean that the patient will have any interest in participating in that trial. From the patient perspective, whether or not a trial is suitable depends on far more than merely whether or not the patient is eligible. Patients are concerned about how much time and effort will be required for them to participate in the trial (for example how many site visits), what is the distance of the patient home to the trial, what kind of medical interventions the trial might involve, whether the trial carries any risk, etc. Here we assume that overall trial suitability has two components:

1. The probability P(E^{t}|g) that the patient is eligible for the trial given the information g available about the patient. Whilst certain information about the patient could definitely rule out some trials (probability equals zero), because only partial information is available, the patient is eligible for other trials probability less than 1.

2. The probability P(W^{t}) that the patient would be willing to participate in the trial (which we call trial suitability). This in turn is a function of several aspects of the trial: perceived quality of research and perceived benefit to science, perceived benefit to patient, perceived risk or inconvenience to patient, etc.

Combining these two, overall suitability is given by:

P(P)=P(W^{t}|d^{t})P(E^{t}|g) (10)

Willingness to participate (i.e. another way of expressing trial relevance or suitability) is difficult to predict, however it is clear that some aspects of the trial (such as geometric distance of the trial site from the patients home) are strongly correlated. We proceed by developing simple parametric models and then learn the parameters from real data about which trials patients participate in. For example, a reasonable model of a patient's willingness to travel to more or less distant trial sites could be expressed as a distance discount function as possible:

P(W^{t}|d^{t})=1−e^{-d}^{^{t}}^{/d}^{₀} (11)

where d^{t} is the distance of the nearest site for trial t from the patient's home and d₀ is a parameter that governs how much less willing the patient is to travel to the trial site as distance increases.

Results are displayed to the patient with the most relevant results first in the manner of a search engine. As the patient answers more questions, the results will be re-ranked as a more complete picture of the patient is built up.

The suitability of a trial is a complex model of the various attributes of the trial and it may also be extended to more general measures. Suitability may also take into account the patient friendliness of the trial. Suitability may be a function of how invasive the medical procedures in the trial are, or whether there is car parking, or if the trial sponsor has attached a document to explain clearly what it is about, or it could also be why the trial matters to society. Trial suitability may also take into account of various other factors that determine how likely a patient is to participate in a trial for which he is eligible, e.g. the distance he is willing to travel to the nearest trial site, or the nature of the interventions. Hyperparameters of such a model (e.g. the discount used to penalise more distant trials) may be learnt by monitoring whether or not patients go on to participate in trials.

It is therefore possible to hypothesise the general form of the suitability model without knowing the value of its attributes. Machine learning approaches are in this case used to predict the suitability model.

What happens after patients have been matched to clinical trials is also critical, such as knowing what happens after the patient has been matched, whether the patients have participated in the trial, and how satisfactory they have found the trial. These follow up attributes will also be inputs of the suitability model to improve the ranking algorithm and the effectiveness at matching patient to trial sponsor. This data is of substantial potential value.

### 3.5 Screenshots Examples of MATCH

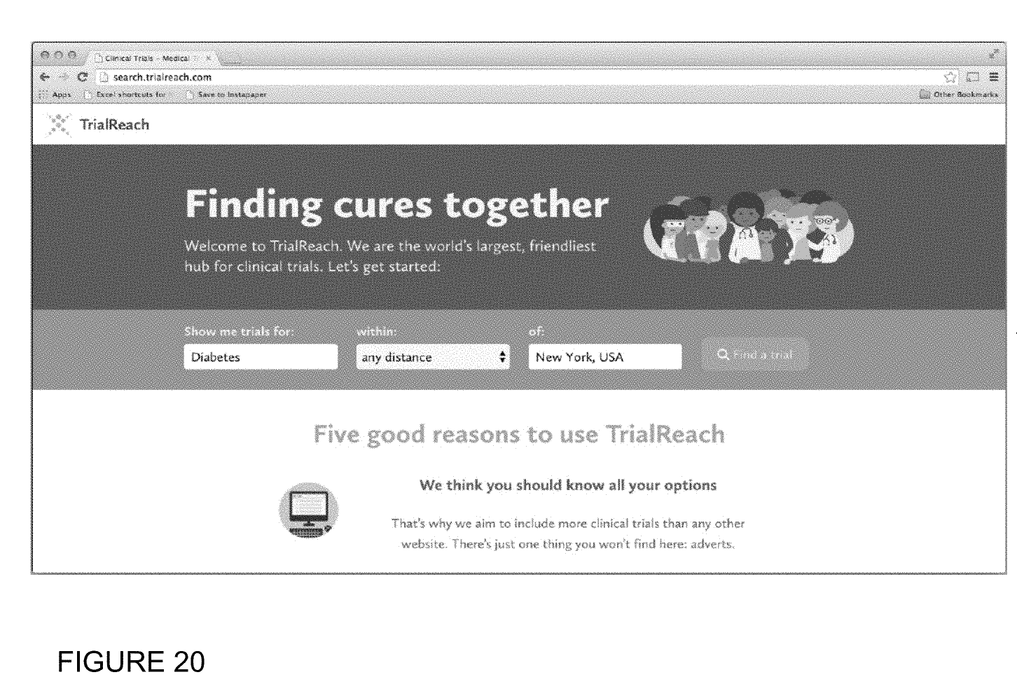

FIGS. 20 to 28 are screenshots that show examples of the patient facing web interface: MATCH.

FIG. 20 shows a web interface example where the patient can enter the condition for which a trial is needed, and is able to select an acceptable distance from the trial centre to an entered city or area or postcode.

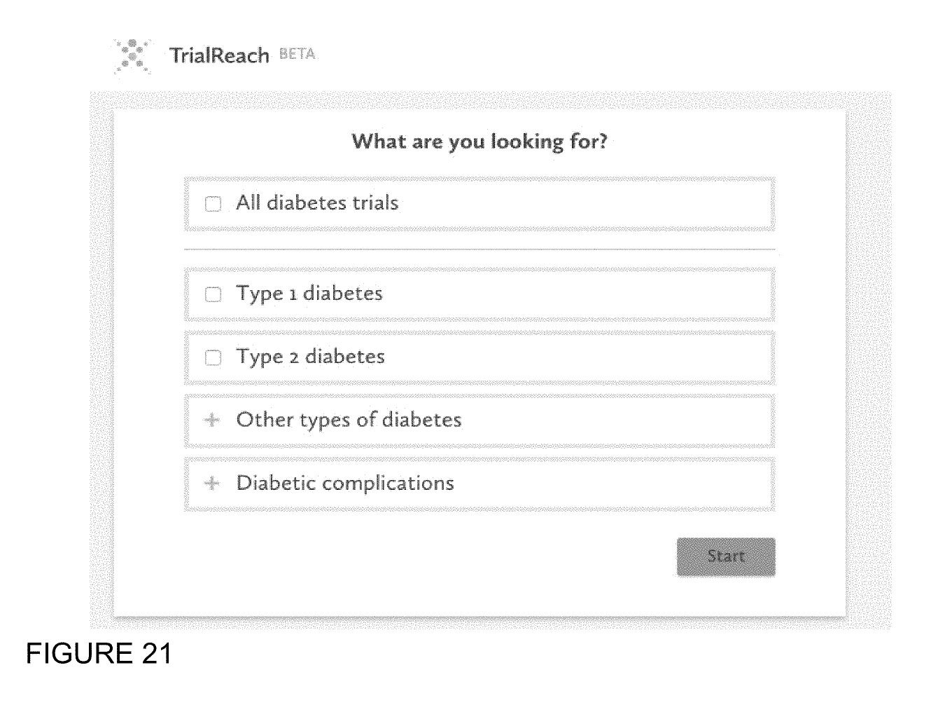

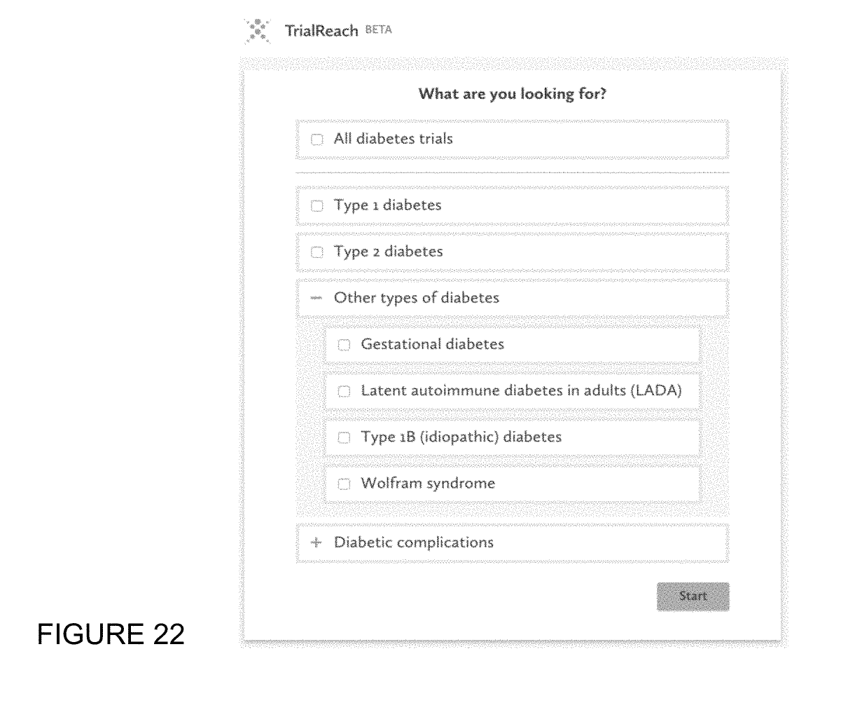

FIG. 21 shows a web interface example where a patient is looking for a diabetes trial. It shows a combination of static and dynamic questions. FIG. 22 shows a dropdown menu available via one of the dynamic question as displayed in FIG. 21. The dropdown menu lists even more specific conditions in order to clarify the intent of the patient enquiring for a trial.

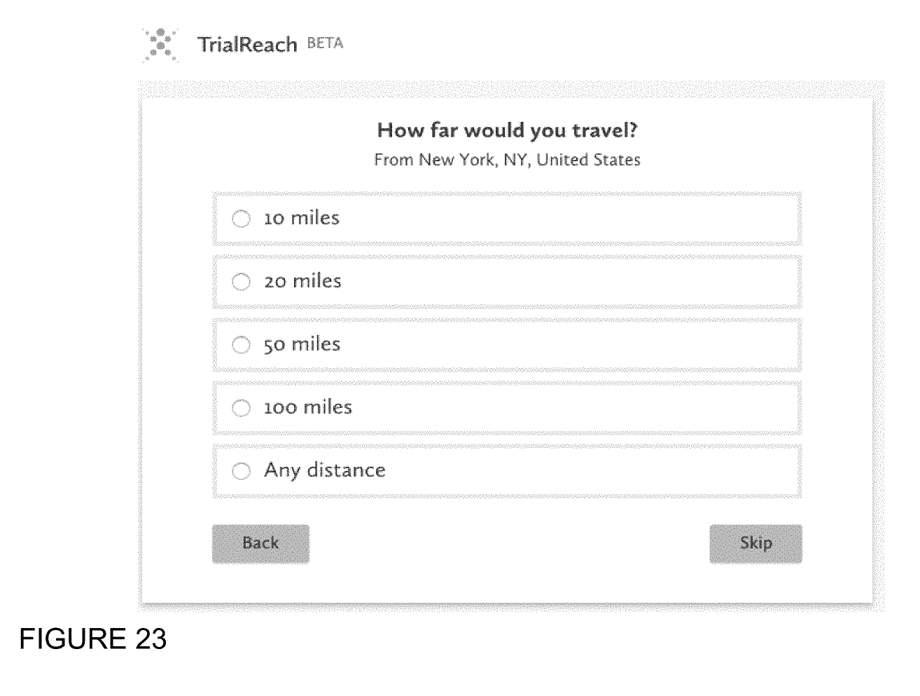

The system dynamically generates the next question as shown in FIG. 23 in order to help filter down a list of trials within the chosen distance area. A ‘Back’, ‘Next’ or ‘Skip’ button may be available.

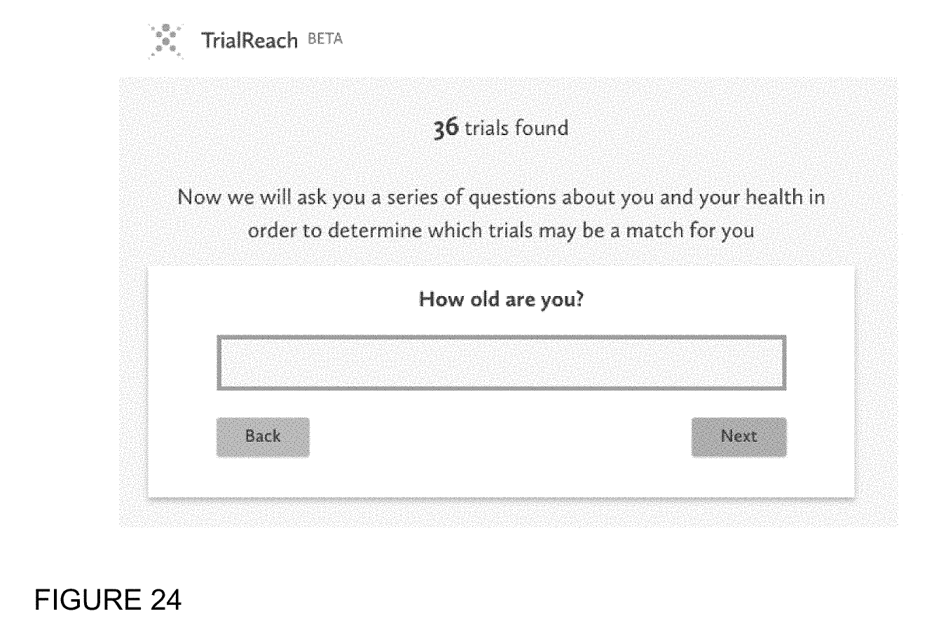

The answers of the questions can be either selected from a multiple-choice answer form or typed as seen in FIG. 24. A count of the number of possibly suitable trial may also be generated and displayed.

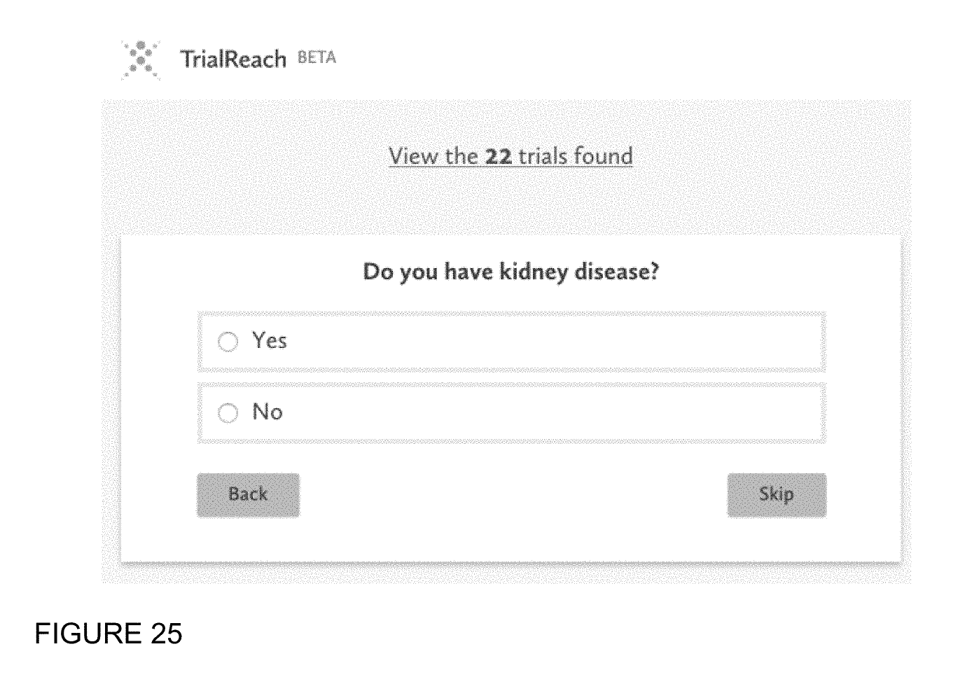

Further questions are generated as shown in FIG. 25 as the system continues to narrow down the trials that the patient could be eligible for and excludes the one for which the patient is not eligible for. The count of trial goes down as the questions are answered.

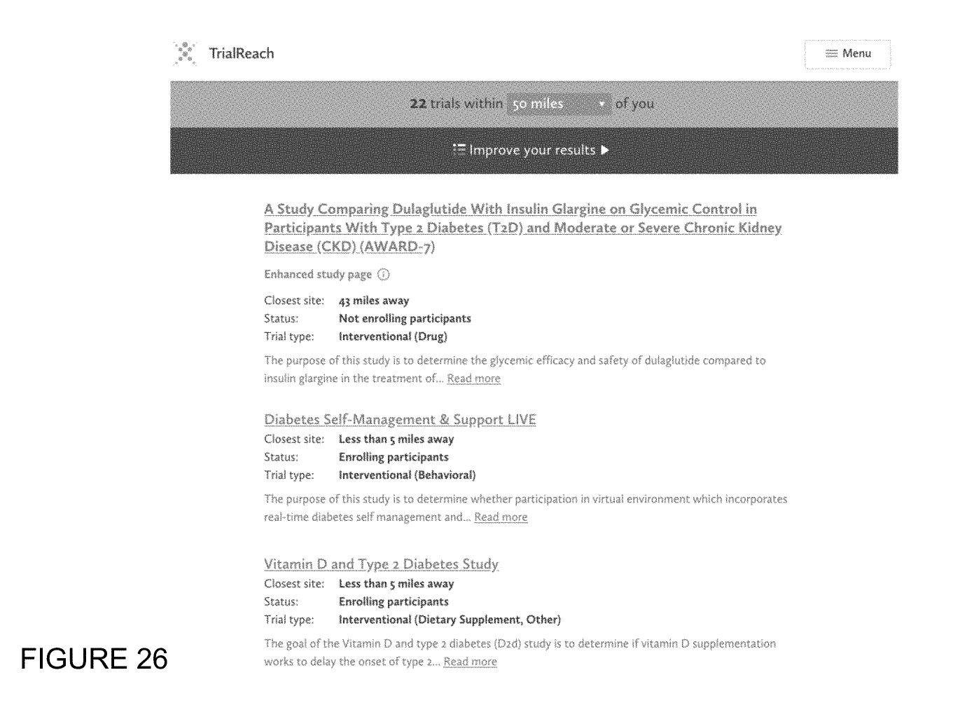

A patient may choose to view the number of suitable or relevant trials as shown in FIG. 26. A list is displayed with all the suitable or relevant trials that match the results of the questions that have been answered so far.

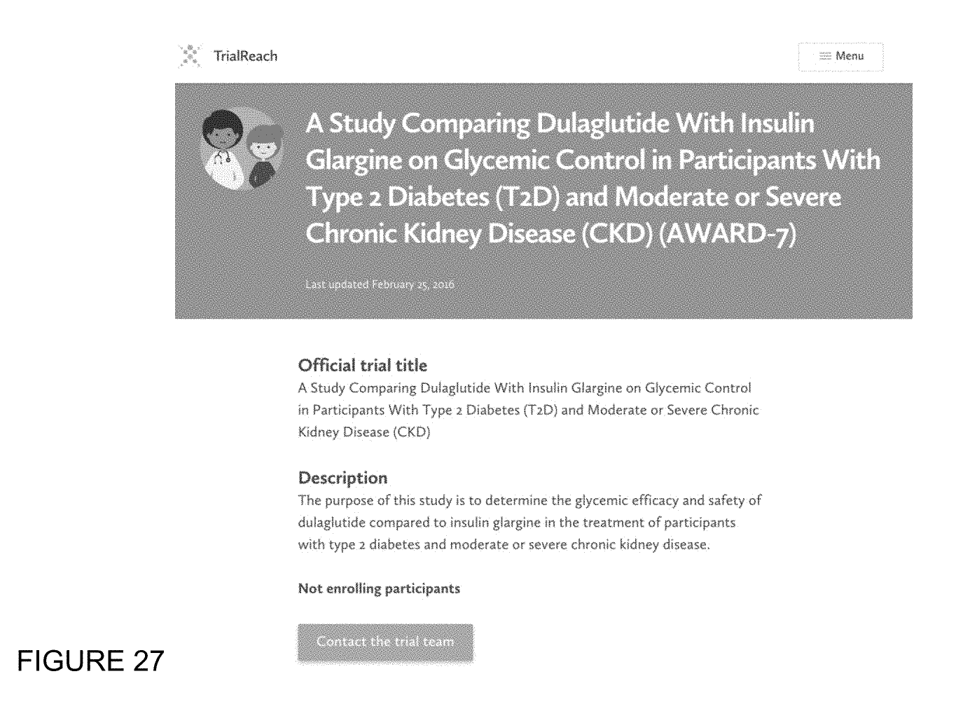

FIG. 27 shows an example of a study page displaying all the details of a particular trial.

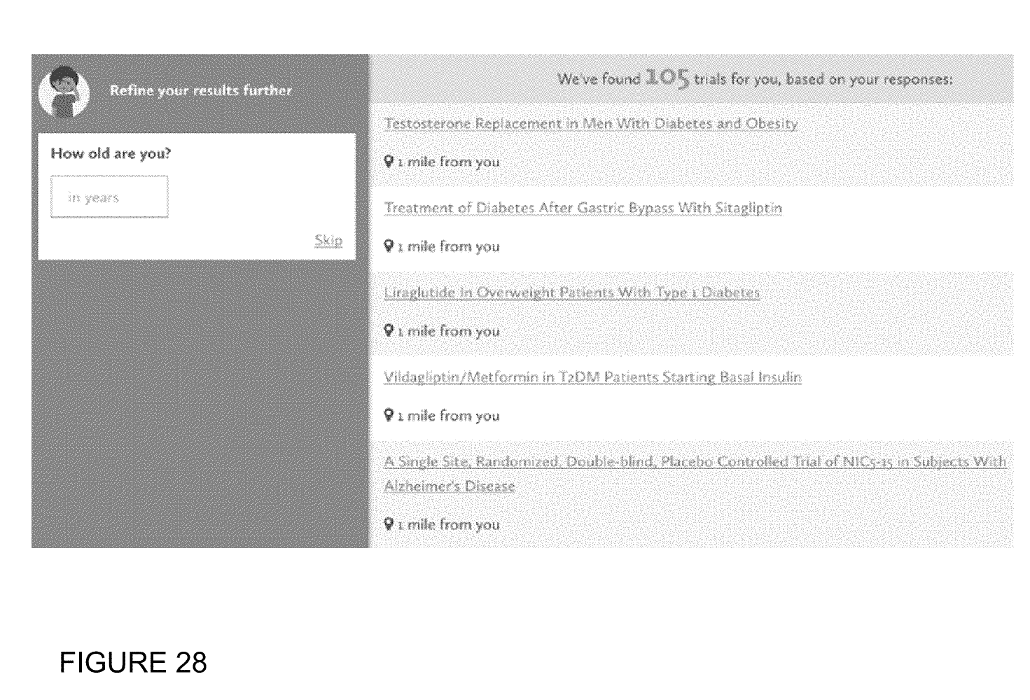

FIG. 28 shows an example of a window view that is split into two different sections. The right hand side lists all the suitable or relevant trials that match the results of the questions answered so far and the left hand side shows the next question for which a new answer can be entered. The list of the questions that have already been asked may also be displayed with their respective answers with an option to check and/or correct previous answers.

### Section 4: DATA

### 4.1 Patient Data Collection

A stream of valuable information or attributes is continuously harvested as patients interact with the web UI. Patients may also opt to register their information through the website or through a third-party such as with a healthcare provider for example. A profile is created for the registered user, which can be modified or updated by the user. The information provided can be personal details including but not limited to information about medical history, demographics, and others. Browsing habits can also be collected in the form of “cookies” or “internet tags” for example. Geographical location may also be derived by collection IP addresses.

The vast majority of the data may also be anonymous. However, anonymous data is collected even for patients that leave the web UI without logging in or completing the forms. (For example a person with diabetes in Florida that might enter the web site to look for trials in a selected area and leave the site). The data collected may still be of value—for example, the aggregated data might indicate that there are many people in Florida with diabetes, and that is in itself relevant information for pharmaceutical companies, for instance.

Hence, registered information as well as anonymous information is continuously harvested and collected on an aggregated form. Additionally, surveys might also be sent to patients with the goal to fill up the gaps in the collected data.

Furthermore, data collected may also include the relevant TAG concepts in order to allow for a structured analysis of the data. Patient data may also be inferred using the rules for logical and statistical inference described previously.

This continuous stream of data presents extremely useful information that can lead to extremely valuable discovery. For example, the system may learn which questions a patient can be expected to know the answer to, and which questions patients often answer mistakenly. The system may then also validate or cross-check its learning by asking the same question expressed in two different ways. For example, some medically complicated criteria might be quite incomprehensible for most patients. On the contrary, other technically difficult concepts might be easily understood for a targeted group. The discoveries may be for example the list of incomprehensible criteria and the list of easily understood criteria. As an example, most people with diabetes understand what HBA1C is and also know their own measurement value, as they have to monitor it carefully.

Hence, all of the harvested data is continuously stored, monitored, analysed and used to improve the ontology as well as probabilistic models. Furthermore, a timestamp may be added to the harvested data when collecting patient's data as it may be critical for some various conditions for example.

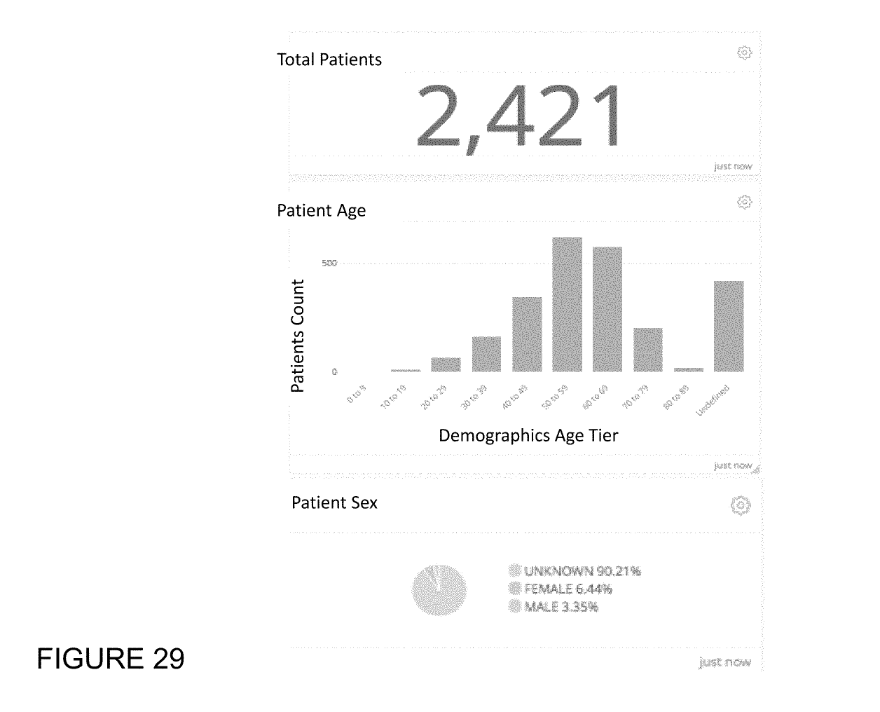

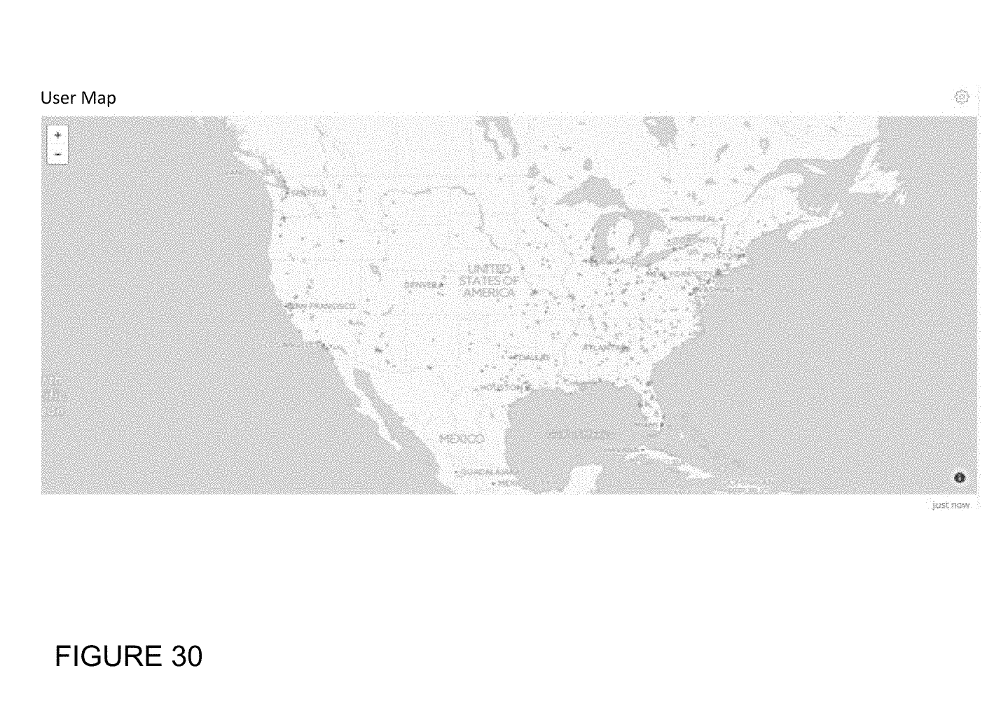

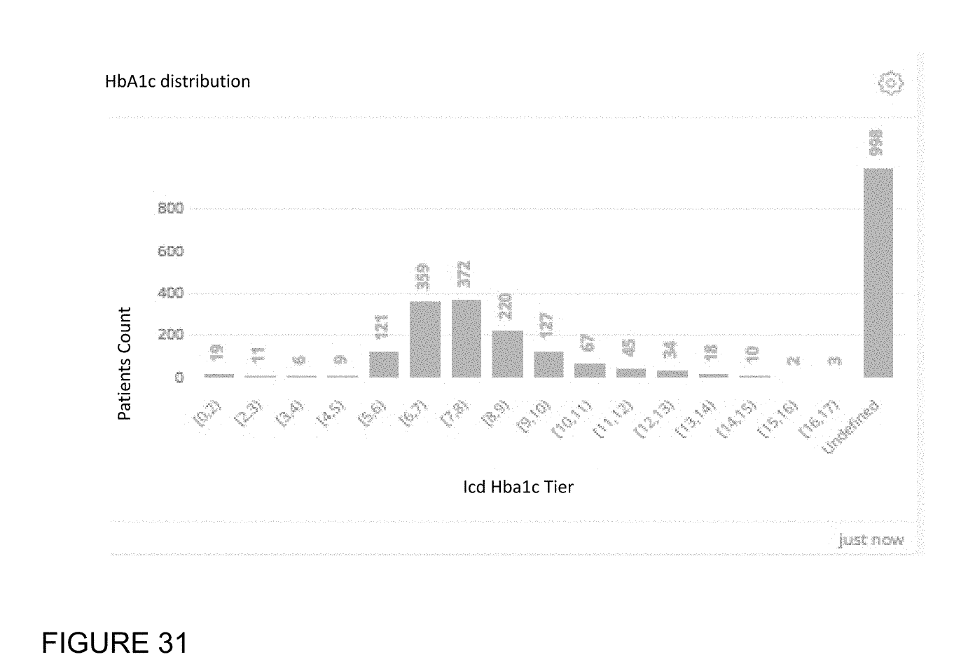

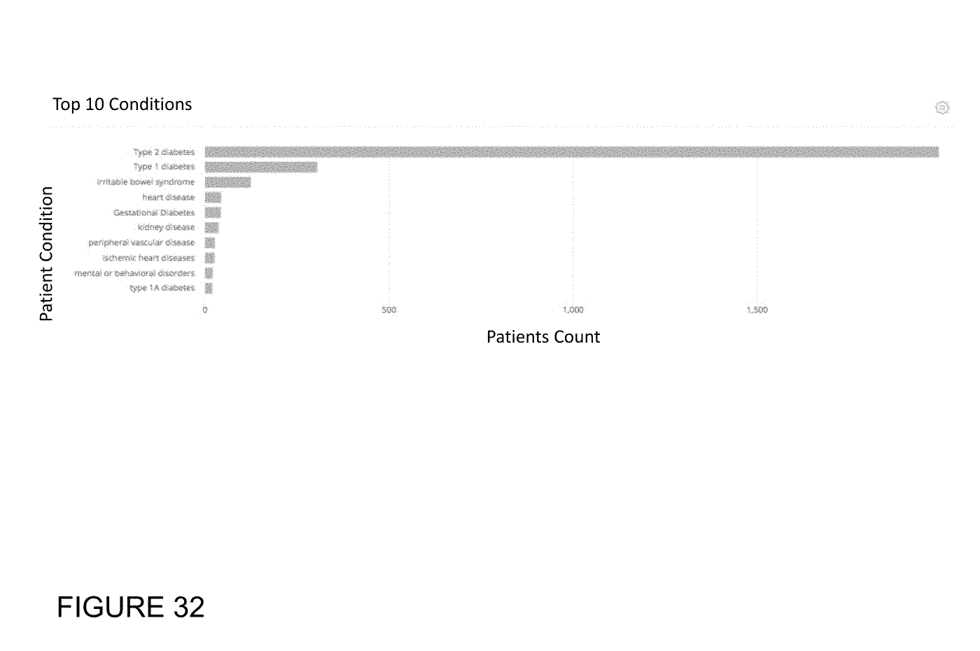

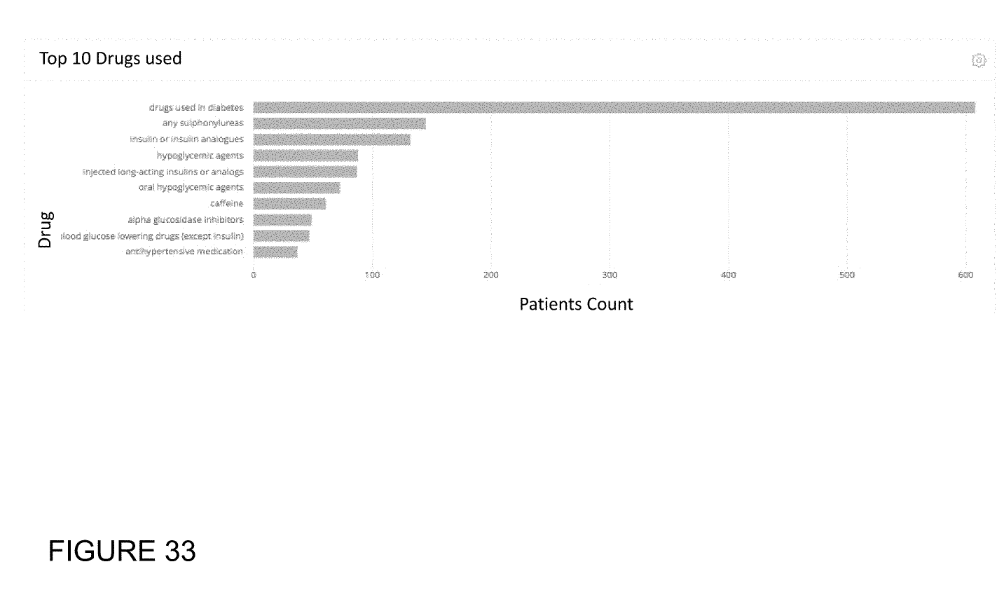

FIGS. 29 to 33 show screenshots of a dashboard allowing one to automatically view and analyse the data as it is being collected through the query based clinical trial matching system. Key metrics of the demographic makeup of the patients using MATCH may be displayed and analysed dynamically such as the total number of patients, the breakdown patients versus age range, and the percentage of female or male, as seen in FIG. 29. FIG. 30 shows location demographics with a map displaying the location of the users within the USA of MATCH. FIG. 31 displays an histogram of HbA1c distribution per number of patients. FIGS. 32 and 33 display the top 10 conditions and the top 10 drugs used respectively.

In addition, individual patient profiles may be built up from the answers they have given, and it may be possible to alert them as new trials have become available for which they may be eligible.

### 4.2 TrialManager

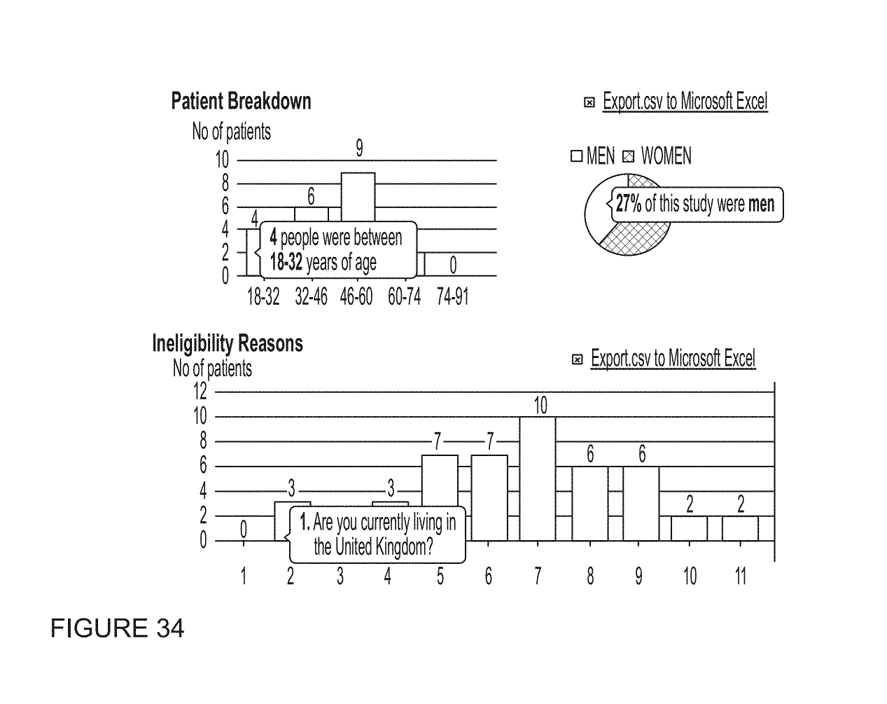

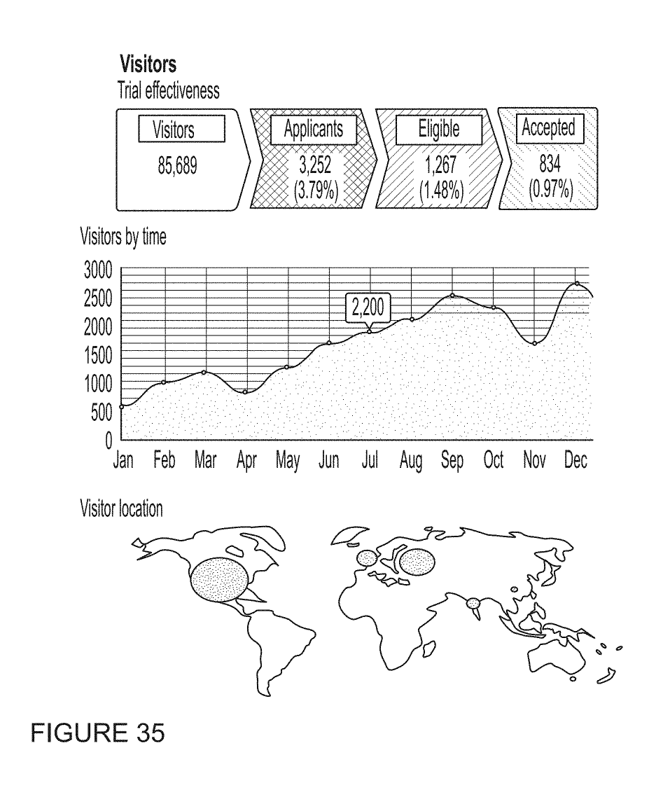

TrialManager is a dashboard through which sponsors can view key metrics relating to a particular Study, as shown in FIGS. 34 and 35:
Numbers and geographical location of visitors to the Study Page;
Numbers, age and gender of people who complete the pre-screener;
Numbers, age and gender of eligible candidates and approved referrals (as described in Section 6.1);
Percentages of ineligible candidates failing on each question of pre-screener (if an advanced study-specific pre-screener is selected);
Progress of referrals across the trial by stage from new referral to randomized as provided by the sites via the Site Portal.

### 4.3 Design and Operation of Trials

Clinical trial protocols are often designed with the clinical aspects in mind without giving much regard to the challenges of candidate recruitment. A system, which uses the continuously harvested data, is developed to improve the planning of clinical trials.

In particular, a dynamic system is developed such that when a clinical trial criterion is entered, the population the trial may be able to target is predicted and displayed via a heat map for example. This is done through accessing in real time the database of harvested data. Displayed information includes for example the possible trial sites with corresponding locations and size of the population. The estimated cost of a particular trial is also generated and displayed along with predicted attributes of the targeted population. Estimated speed and cost to recruit may also be displayed.

Additionally, the system is able to predict further valuable information dynamically, such as by what amount the targeted population will increase or decrease when changing a particular criterion. As an example, the designer of the clinical trial protocol might enter a criterion such that the candidates must not have smoked for the past 6 months. The interactive system is able to inform the trial designer that if he was to reduce the requirement to patients that have not smoked for the past 3 months, it may then be twice as easy to recruit candidates for the particular trial.

The system can also provide further data on specific attributes that are common to a population. As an example, this amount of population is on Facebook, or might be likely to respond to an email, or prediction on how willing they are to travel.

Information is available on which trial should be stopped because it would not yield a big enough sample size of suitable candidates. Accordingly, information is available on trials that are most likely to yield a big enough sample size of suitable candidates.

Additional information that is also available relates to the potential drugs that need developing or what sort of research for which condition is needed and their expected targeted population size and details.

The system also helps to educate the trial designer to include critical details that might not always be obvious, such as for example logistics details (parking is available, overnight accommodation is possible).

The output of the system is a structured clinical trial protocol wherein multiple representations are possible, for example a patient-friendly representation wherein clinical trial protocol details are easily understood by potential candidates and where nonclinical trial protocols details are also given.

The goal is to provide an industry standard tool for all clinical trial protocols—e.g. eligibility criteria normalised across multiple clinical trials, so that we can efficiently compare data across different trials and join data across different trials. Ultimately as new trials come out, they will not need translations if they are created using TAG.

The tool may have one or more of the following features, but not limited to:
a view of the available number of patients for a given trial, given its eligibility criteria, with the ability to project how long it will take and how much it will cost to find patients for a trial;
a view of the impact of each individual eligibility criterion on the addressable patient population, the projected rate of recruitment, and the cost per patient;
a view of the geographic location and density of patients meeting certain criteria, and automatic selection of the optimal trial sites; this may help to determine how many trial sites there should be, and where they should be located (given the density of patients and their propensity to participate);
a guide to how best to find patients for a trial, and what blend of approaches will be optimal for cost and speed (e.g. direct contact, asking physician, sponsored advertising, outreach via partner groups, social media);
a quantitative view of the impact of different explanations and messages on the rate of patient recruitment;
a view of the potential skew of a patient sample according to the means of finding the patients comparing the sample it to the general population for a disease;
a view for patients of which trials a potential course of treatment they may be considering would exclude them from
a view of the success of individual trial sites in recruiting patients, based on the available patients in their area, and on the ultimate number of patients who join a trial. This might depend on factors such as how welcoming the facility is; the quality of the staff and the information they provide; the timeliness with which they contact patients;
market sizing tools to help with strategic investment decisions by understanding patient demand especially for correlations between patient attributes (i.e. more complex than simple incidence data);
a tool for viewing information on “competing trials” i.e. as a sponsor I would like to know which other sponsors are recruiting for the same kinds of patients as me;
benchmarking of recruiting rates on similar/competing trials i.e. “are my competitor trials recruiting faster/slower than me” (in aggregate/anonymised);
a view for patients of which trials are the hardest to fill and hence where they could be of greatest benefit to research, by signing up for those ‘difficult’ trials if they are eligible.
a tool to allow sponsors to add a custom question for the query-based clinical trial matching system for one or multiple clinical trials.

### Section 5: Patient Trial Matching Using Electronic Health Records

A system to match clinical trial using an individual's EHR is developed. The system may also perform bulk matching of many EHRs against a set of trials.

Our approach to patient-trial matching using information obtained from Electronic Health Records depends on a number of important innovations:
We cast patient-trial matching as a many-to-many problem, where all candidate patient-trial matches are ranked according to a measure of their quality. By this means, we can prioritize patient-trial pairings for efficient further investigation. The average cost of recruitment is also therefore reduced.
A related innovation is the possibility of assigning a different importance weighting to different trials e.g. on the basis of the impact of the disease targeted by the trial on quality-of-life measures for the patient population. Thus we can prioritize patient-trial matches in such a way as to achieve a desired trade off between benefit to individual patients and benefit to medical research.
Most existing approaches to patient-trial matching treat the question of whether a patient satisfies the eligibility requirements for a trial as one with a straightforward yes or no answer. However, this approach doesn't account properly for the uncertainty associated with patient information obtained from real EHRs. By contrast, here we model patient eligibility as a probability. We assign to each candidate patient-trial match a probability of eligibility that properly reflects the uncertainty inherent in the information we have about the patient's medical history. Uncertainty arises because of (i) the risk of making mistakes in automatically derived interpretations of unstructured medical data, (ii) gaps in the patient's EHR, and (iii) when making uncertain inference about an individual patient using statistics derived from a larger body of patients (and see below).
EHRs provide information about aspects of the patient's medical history (‘patient attributes’). For example, the EHR might allow us to infer that the patient has taken a particular drug, or has had a particular disease for a given amount of time. But there may be considerable uncertainty inherent in automatically obtained interpretations of EHRs, for example because NLP algorithms are used to extract information from unstructured text fields. Therefore, a useful innovation is to model the uncertainty inherent in our interpretation of the EHR using appropriate probability distributions. For example, our interpretation of a Boolean valued patient attribute might be modeled as an independent random variable with a Bernoulli distribution. This approach allows us to marginalize over the space of possible interpretations of the data when using the EHR to make inference about the patient.
We use information provided by a corpus of patients to create a statistical patient model. Such statistical models may be used to model the conditional probability distribution over an unknown patient attribute given some other patient attributes. However, in the context of EHR matching, a useful further innovation is to model the systematic inaccuracies inherent in EHRs. Specifically, we use real patient data to model the conditional probabilities that (i) certain aspects of a patient's history will not be recorded in his EHR and (ii) certain aspects of the patient's medical history will be recorded incorrectly in his EHR.
Such statistical models may be obtained using data provided by real patients, for example during question-based patient trial matching (see separate patent application) or whilst obtaining additional patient-provided information to supplement that already present in EHRs (see below).
That the patient satisfies a trial's eligibility criteria is a poor predictor of whether a patient will actually go on to participate in that trial, i.e. the outcome that matters most to trial sponsors and patients. In practice, the likelihood of trial participation is a function not only of the patient's suitability to the trial sponsor but also of the suitability of the trial to the patient. The latter might be a function of several aspects of the trial such as whether or not overnight stays are required, whether the patient will have to spend an appreciable amount of time travelling to the nearest trial site, whether the patient might receive a placebo instead of an investigational drug, etc. We learn a composite model of patient-trial suitability that accounts for the needs of patients and trial sponsors and reflects the likelihood that the patient would participate in the trial if presented with the option to do so. The relative importance of different patient concerns is learned by a machine learning approach using data about the participation in trials of real patients.
We introduce a new measure of the quality of a set of ranked pairwise patient-trial matches. This measure takes account of both the suitability of pairwise matches and the fact that the highest ranked pairwise matches will be further investigated first. The measure gives the highest ranked match greater importance than the second highest ranked match, and so on. This is achieved using a suitable rank discount function.
We refine the hyperparameters of our trial suitability model by optimizing our system against a metric that reflects the extent to which our matching engine is effective in facilitating patient participation in trials.
Since information extracted from a patient's EHR may be incomplete or uncertain, a further useful innovation is to augment the information extracted from the EHR with information provided directly by the patient (or his or her doctor). One strategy for doing this is to direct questions to patients via a software user interface. However, since a patient's budget of enthusiasm for answering questions is limited, it is important to ask maximally informative questions first.
We use a measure of the expected informativeness of patient-provided information to prioritize the questions we put to the patient. Our informativeness measure reflects the expected increase in the quality of the set of matches evaluated using the quality measure described above. By extracting a confidence measure associated with information extracted from an EHR, we can identify which questions to put to the patient in order to provide greatest improvement to the quality of the set of patient-trial matches.
   Section 6: Electronic Health Record collaboration

Multiple sources may be used to gather information or attributes for a particular patient. These are but not limited to:
Electronic Health Records (as well as EMRs, electronic medical records)—hence in the US, Blue Button users can share their health records for automatic matching with potentially suitable clinical trials.
Web UI of the present invention: TrialReach.com.
Physicians visits.
Hospital visits.
Data shared to an authorised third party.
Other web facing products such as for example patient interest support website group.
Surveys (online or offline).
Apple Healthkit.
Wearables or any type of sensor devices (for example blood screening kit, or any other homekit gathering patients data).
Any other electronic health devices or services.

A novel aspect of the invention is to structure all of the information that can be gathered from multiple sources and combine it together in order to find a clinical trial match more efficiently.

For example, MATCH may be integrated with observational study products, such as health applications on smartphones. Since the smartphone application users may consist of engaged patients for a given condition, it may provide a good source of engaged patients willing to participate in clinical trials.

Furthermore, the system may also update or correct patient's electronic health records. Electronic health records tend to focus on medical information, for example drugs, disease, or treatment. Other attributes that might be relevant to a clinical trial such as for example life style questions (Do you smoke a lot at the moment?, are you overweight?, is a carer accompanying you?) might not be recorded in electronic health records.

Some answers may benefit to be provided from one source rather than another. For example, a question such as are you pregnant? is best to ask patients directly rather than to extract the answer from the electronic health record. Whereas for a question such as are you taking this particular drug?, it is best to extract the answer directly from the electronic health record.

In addition, recruitment for clinical trials can prove more difficult for some conditions than for others. As an example, for a clinical trial for diabetes it can often be relatively easy to find candidates, whereas a clinical trial for cancer might prove more difficult. As a result, it sometimes might be critical to involve physicians in the process of clinical trial matching.

Thus, a tool has also been developed that can be integrated with the physician workflow, such that the physician is alerted when a clinical trial is taking place in a certain area. Physicians (eg oncologists) may view trials relevant to their patients, and answer specialist medical questions requiring knowledge or expertise the patient may not have to help refine the matches (this may constitute a third source of information, in addition to asking the patient and inspecting the EHR). Physicians may be alerted in real time that the patient they are talking to or treating is potentially eligible for a clinical trial in their location based on data entered into the EHR system. During the physician visit, the patient is able to answer further questions from the physician in order to assess the suitability of the trial. The physician can in effect suggest or ‘push’ possible trials directly to his or her patients. The physician may also has the ability to launch immediately into prescreening questions to book the patient in for a screening visit if they match the initial criteria.

An interface may be available for physicians (e.g. oncologists) to view trials relevant to their patients, and answer specialist medical questions requiring knowledge or expertise the patient may not have to help refine the matches (this may be a third source of information, in addition to asking the patient and inspecting the EHR.)

Equally, a patient may subscribe to an automated service that would push potentially suitable clinical trials to him or her, without the need for any prior completion of an eligibility survey by the patient.

### 6.1 Referral Management Overview

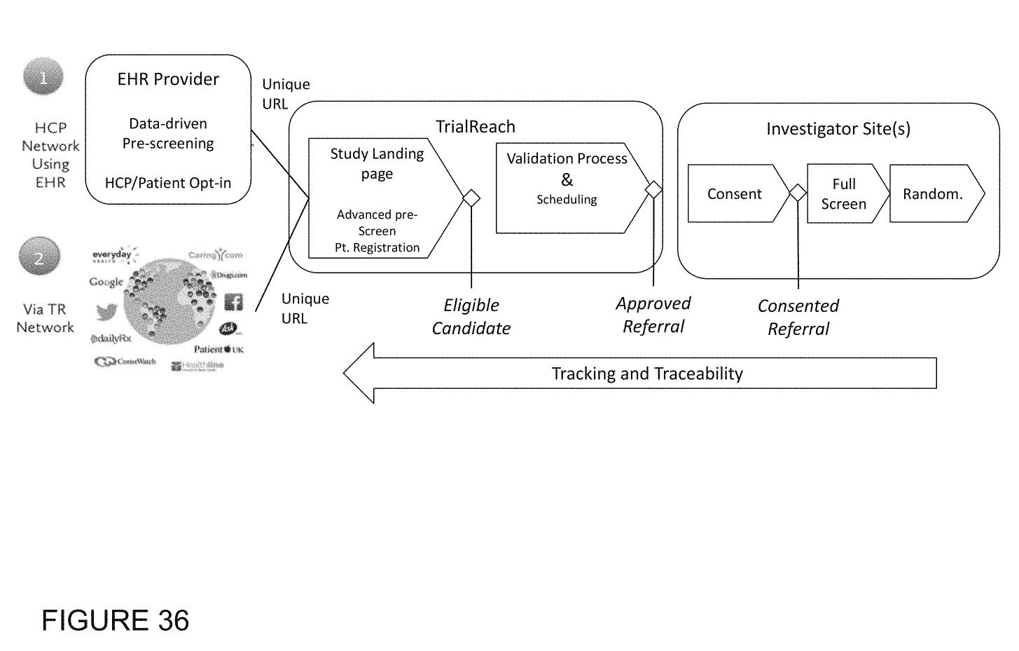

FIG. 36 is a diagram that summaries a referral management patient flow for a EHR provider collaboration.

A key for recruitment success is assisting an interested patient to follow-through with site visit for full screening, consent and enrollment. Our Referral Management services support this “last-mile” conversion through multiple stages of the process, including:
Patient validation.
Medical pre-screening (optional).
Direct booking of patients into sites calendars.
Site follow-up and tracking analytics.

Patient validation: each patient that passes the study pre-screener is contacted by a TrialReach representative to review and validate his or her answers as well as confirm the patient's interest to move to the next step. This personal human-to-human touch is critical for patients and for avoiding “false positives” patients being sent to sites. The sites receive only the patients who have been vetted and remain interested in the study. The sites appreciate this process as it also lowers the overhead and burden of their operational personnel.

In the case of an EHR provider collaboration, the patient would have initial data-driven pre-screening via analysis within the EHR system. Through their health care provider (HCP), they would opt-in to next steps, specifically a link out to a study page. This study page may have a complementary pre-screener for study specific questions not answerable through data (e.g. “would you be willing to . . . ” type of questions). The page is also a registration page for contact information and next steps of the process.

Medical pre-screening (optional): Through partnership (such as with Topstone Research for example), thorough medical pre-screening is offered. If chosen, the medically qualified agents prescreen patients on the basis of the entire protocol, thereby sending only very highly qualified patients to sites. This is an optional advanced validation process that is most commonly selected where a study has complex eligibility criteria or medical discernment is necessary. In the case of robust HCP interaction by the patient at point-of-care, this optional service may not be necessary.

Direct booking of patients into sites calendars: TrialReach operation teams coordinates with patients to set appointments for patients at the investigator site. This reduces site workload relating to calling each patient and scheduling them in, minimising referral wastage.

Site follow-up: TrialReach staff stay in close contact with the sites. Through the Site Portal tool, we are able to track patients and provide valuable insights to the patient engagement process. Where necessary, we follow-up with sites to ensure they are engaging patients and completing the screening and consent process.

### 6.2 Tracking and Tools for Premium Services

Throughout the Referral Management process we track the sources and flow of patients.

The tracking is critical for our partner network revenue sharing business model. Through the use of unique referral URLs, we are able to identify the source of the patients and once registered on our site, we track them through to the investigator site, including through to consented and enrolled if requested by the sponsor.

For the sites and sponsors, we provide online tools to see and measure progress of patient engagement and study enrollment.

### Site Portal

The Site Portal is a secure portal through which sites receive and can manage referrals. This is the primary coordination system for patient management. The site and TrialReach are able to view:
Patient contact details (All patient contact information is managed through our standard privacy controls and is blinded to sponsors following ICH and GCP norms.)
Completed pre-screener
Manage the status of candidates from new referral to randomized

### Note

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred example(s) of the invention, it will be apparent to those of ordinary skill in the art that numerous modifications can be made without departing from the principles and concepts of the invention as set forth herein.

1. A computer implemented method for interactively matching a patient with suitable or relevant clinical trials and dynamically displaying a list of matching trials on a graphical user interface (GUI), the method comprising:
(1) receiving, from a database, a set of clinical trials' eligibility criteria,
(2) presenting the patient a series of question(s) on the GUI, generated by a probabilistic query-based clinical trial matching system,
(3) receiving, via the GUI, the patient's answers to the series of question(s),
(4) modeling patient eligibility to the clinical trials as a probability based on the patient's answers, in which the probability of eligibility is calculated by the probabilistic query-based clinical trial matching system measuring or calculating trial suitability or relevance, wherein trial suitability or relevance is defined as a likelihood that a patient will participate in a trial, and
(5) automatically filtering and ranking a list of matching trials, by the probabilistic measuring query-based clinical trial matching system, based on the calculated probabilities,
in which, as the patient answers more questions, the list of matching trials is dynamically filtered and re-ranked by the probabilistic measuring query-based clinical trial matching system as a more complete picture of the patient is built up, and the GUI automatically displays the list or a sublist of dynamically filtered and re-ranked matching trials from the highest ranked matching clinical trial at the top to the lowest ranked matching clinical trial at the bottom.

2. The method of claim 1 in which, the highest ranked matching clinical trial is the clinical trial for which the patient has the greatest likelihood of participation.

3. The method of claim 1, in which trial suitability or relevance is a function of the probability that the patient meets the eligibility criteria for the trial given the information received, via the GUI, from the patient and of the probability that the patient would be willing to participate in the trial.

4. The method of claim 1 in which ranking does not only depend on whether or not the patient is eligible or meets the eligibility criteria of a clinical trial.

5. The method of claim 1 in which ranking is a function of how much time and effort will be required to participate in the trial including one or more of the following: number of required clinical trial site visits, requirement of overnight stays, distance of the patient home to the site, travelling time to the site, availability of car parking for the patient, the medical interventions involved in the trials, how invasive the medical procedures in the trials are, whether the trial carries any risk, whether a placebo instead of an investigational drug might be received, whether the trial requires abstinence from food or drink or other activities, or if the trial sponsor has attached a document to explain clearly what the trial is about.

6. The method of claim 1 in which ranking is a function of which of the trials will be further investigated first.

7. The method of claim 1 in which ranking takes into account patient concerns learned by a machine learning approach using data about the participation in trials of real patients.

8. The method of claim 1 in which ranking takes into account patient behavior learned by a machine learning approach using data about the participation in trials of real patients.

9. The method of claim 1 in which ranking takes into account the impact of the disease targeted by the trial on quality-of-life measures for the patient population.

10. The method of claim 1, in which ranking is based on a desired trade off between benefit to individual patients and benefit to medical research.

11. The method of claim 1, in which, as the patient answers more questions, an updated count with the number of matching trials is dynamically generated and displayed on the GUI.

12. The method of claim 1 in which questions are dynamically generated so as to reduce the number of trials for which the patient is definitely not eligible as quickly as possible.

13. The method of claim 1, in which questions are dynamically generated so as to increase the expected increase in standard search engine scores such as normalized discounted cumulative gain.

14. The method of claim 1, in which the method includes the step of receiving a search query by the patient including the condition for which a trial is needed.

15. The method of claim 1, in which the method includes the step of receiving a search query by the patient including the chosen travelling distance area from the patient home to the clinical trial.

16. The method of claim 1, in which the questions that have already been answered by the patient are displayed alongside the patient's answers to those questions on the GUI.

17. The method of claim 1, in which the patient is given the option to check and/or correct previously answered questions on the GUI.

18. The method of claim 1, in which the list or sublist of matching trials is displayed alongside the subsequent question to be answered on the GUI.

19. The method of claim 1, in which, when a question is presented, the patient is presented the option to answer the question or to click on a ‘back’, ‘next’, ‘unknown’ or ‘skip’ button.

20. The method of claim 1, in which the matching trials presented on the GUI are associated with a link enabling the user to access a page with information on a clinical trial's eligibility criteria.

21. The method of claim 20, in which the information on a clinical trial's eligibility criteria has been automatically summarised for facilitating the understanding of the patient.

22. A system for interactively matching a patient with suitable or relevant clinical trials and dynamically displaying a list of matching trials, the system comprising:
(1) a database for storing a set of clinical trials' eligibility criteria,
(2) a display device for presenting the patient a series of question(s), generated by a probabilistic query-based clinical trial matching system,
(3) a user input device, for receiving the patient's answers to the series of question(s),
(4) the probabilistic query-based clinical trial matching system for (i) modeling patient eligibility to the clinical trials as a probability based on the patient's answers, in which the probability of eligibility is calculated by measuring or calculating trial suitability or relevance, wherein trial suitability or relevance is defined as a likelihood that a patient will participate in a trial, and (ii) for automatically filtering and ranking a list of matching trials, based on the calculated probabilities,
in which, as more answers are received from the user input device, the list of matching trials is dynamically filtered and re-ranked by the probabilistic query-based clinical trial matching system as a more complete picture of the patient is built up, and the display device automatically displays the list or a sublist of dynamically filtered and re-ranked matching trials from the highest ranked matching clinical trial at the top to the lowest ranked matching clinical trial at the bottom.
